(19) United States
(12) Patent Application Publication
(10) Pub. No.: US 2022/0165384 A1
(43) Pub. Date: May 26, 2022

(54) BLOCKCHAIN SYSTEMS AND METHODS FOR REMOTE MONITORING

(71) Applicant: NEPHRON PHARMACEUTICALS CORPORATION, West Columbia, SC (US)

(72) Inventors: Hank JIBAJA, West Columbia, SC (US); Jack E. Neil, West Columbia, SC (US)

(73) Assignee: NEPHRON PHARMACEUTICALS CORPORATION, West Columbia, SC (US)

(21) Appl. No.: 17/441,952

(22) PCT Filed: Mar. 20, 2020
(86) PCT No.: PCT/US2020/023818

§ 371 Date: Sep. 22, 2021

Related U.S. Application Data
(60) Provisional application No. 62/822,442, filed on Mar. 22, 2019.

(51) Int. Cl. G16H 20/13; G16H 40/67; H04L 9/32; H04L 9/08

(52) U.S. Cl. CPC G16H 20/13; G16H 40/67; H04L 9/3239; H04L 2209/88; H04L 9/0825; H04L 2209/38; H04L 9/3247

(57) ABSTRACT

Methods and systems are provided to generate and obtain data from mobile medical devices such as nebulizers and mobile monitors such as pacemakers and to store provenance parameters for the data in a distributed ledger for use in verifying the source and accuracy of the data. The methods and systems may further be used to reduce readmittance rates at healthcare facilities.

### CROSS-REFERENCE TO RELATED APPLICATIONS

This application claims priority to U.S. Provisional Patent Application No. 62/822,442, filed Mar. 22, 2019, the entirety of which is incorporated by reference herein.

### FIELD OF THE INVENTION

The present disclosure relates to systems, methods, and apparatuses to improve remote monitoring of mobile healthcare devices using distributed ledger and smart contract technology.

### BACKGROUND OF THE INVENTION

Telehealth involves the use of remote medical devices to collect health-related information traditionally obtained at a point of care (if at all), with a goal of providing more responsive patient monitoring and fewer face-to-face consultations (including primary hospital admissions and readmissions) while delivering cost savings. Mobile drug delivery devices such as inhalers, for example, could be configured to provide use-compliance data to healthcare providers who would monitor the data and potentially intervene to improve patient health and reduce risk of hospital admission. The potential benefits are significant. In the case of inhalers alone, between 28% to 68% of patients do not use metered-dose inhalers or powder inhalers consistently enough to benefit from the prescribed medication. Of an estimated $25 billion spent for inhalers annually, $5-7 billion is wasted because of inhaler misuse.

Telehealth solutions could be customized to target legal and regulatory policy objectives. For example, effective Jan. 1, 2018, the Center's for Medicare & Medicaid Services (CMS) began reimbursing fee-for-service clinicians for monthly non-contact review of patient data for eligible patients. For certain diseases, mobile medical devices could be used to generate much of the patient data used in such reviews. As another example, the Hospital Value-Based Purchasing and Readmissions Reduction Program administered by the CMS may subject a healthcare provider to reimbursement penalties if the provider's readmission rate exceeds competitively-determined benchmarks for certain conditions, such as of acute myocardial infarction (AMI), heart failure (HF), pneumonia (PN), chronic obstructive pulmonary disease (COPD), asthma, elective hip arthroplasty (THA), total knee arthroplasty (TKA), stroke, diabetes (DM), hypertension (HTN), dysrhythmia, and obstructive sleep apnea (OSA). Use data for mobile medical devices could be analyzed to determine whether intervention is necessary to increase patient compliance with a course of treatment, and therefore reduce a risk of primary admission, readmission, as well as improve outcome reporting measures (for example measures indicated under the Medicare Access and CHIP Reauthorization Act (MACRA) and the Merit Based Incentive Payments System (MIPS)).

In all cases, viable telehealth applications must provide data integrity commensurate to health, safety, and privacy risks. As the number of use cases grows, the need for highly accurate, authenticated, and predictable data flows will increase. Telehealth approaches to chemotherapy, for example, would have to achieve a level of data integrity and verification traditionally reserved for a controlled hospital setting before healthcare providers would be comfortable with the attendant health risks and legal liability. Alongside these considerations, accurate and trustworthy medical data are essential for billing and payment schemes that are both efficient and fraud-resistant. Accordingly, there is an increasing need to enable verification of both the accuracy and provenance of data obtained from mobile medical devices, both when data is first stored and throughout its lifecycle.

Data privacy and security requirements must also be addressed. For example, the Health Insurance Portability and Accountability Act of 1996 (HIPAA) and implementing regulations impose obligations on covered parties like healthcare providers who access or become custodians of device-generated data. Approaches are needed to manage the data lifecycle to maintain proper ownership, access and control in compliance with HIPAA and other applicable laws.

### SUMMARY

Certain embodiments may provide, for example, methods, systems, services, products, softwares, computing infrastructure and/or devices for generating and/or obtaining data from deployed mobile medical devices (for example nebulizers or pacemakers enabled with cellular radios) with provenance parameters for the data that can be used by healthcare agents (for example doctors, clinicians, auditors, and reimbursement agents) to retrospectively verify the source and accuracy of the data via immutable distributed ledger transactions. In certain embodiments, for example, the verified data may support a reporting capability via a healthcare API platform. In certain embodiments, for example, the verified data may be used to reduce readmission rates, enable reimbursement for healthcare services, or otherwise improve the care of and value provided to patients.

Certain embodiments may provide, for example, a method for processing data from mobile medical devices. In certain embodiments, for example, the method may comprise receiving (for example via a communication pathway comprising a wireless network such as a cellular network or a Wi-Fi network) a network packet (for example an encrypted network packet), the network packet comprising a first digital signature and a dual payload, a first payload of the dual payload comprising de-identified (or anonymous or device-identified) data (for example timing and duration-of-use data from a mobile medical device such as a nebulizer issued to and registered to a patient), a second payload of the dual payload comprising a second digital signature and a hash. In certain embodiments, for example, the method may comprise authenticating the network packet by verifying the first digital signature using one or more signature verifying algorithms in combination with the first digital signature, the dual payload and a member of a list of public keys assigned to authorized data sources (for example a cryptographic device identification key assigned to a mobile medical device) (for example recovering the dual payload from at least the first digital signature). In certain embodiments, for example, the method may comprise using the member of the list of public keys to identify a patient (for example a patient who has been assigned a mobile medical device such as a patient under treatment for COPD who has been assigned a nebulizer). In certain embodiments, for example, the method may comprise inserting the de-identified (or anonymous or device-identified) data into a record for the patient in a HIPAA-compliant database (for example a HIPAA-compliant database maintained or curated by a manufacturer of a mobile medical device or a healthcare services business). In certain embodiments, for example, the method may comprise pushing the de-identified (or anonymous or device-identified) data (or a hash of the data) to a distributed ledger (for example a public or private instance of a distributed ledger such as Ethereum) without any patient identification information.

A. In certain embodiments, for example, the method may further comprise: authenticating the hash by verifying the second digital signature by passing the second digital signature, the hash, and the member of the list of public keys through one or more signature verifying functions (for example one or more cryptographic functions) (for example by recovering the hash from the second digital signature using the member of the list of public keys). In certain embodiments, for example, the member of the list of public keys may be assigned to a mobile medical device (for example a mobile medical device such as a nebulizer. In certain embodiments, for example, the data may comprise at least one timestamp and at least one duration-of-use for the mobile medical device.

B. In certain embodiments, for example, the de-identified (or anonymous or device-identified) data may be pushed to the distributed ledger from a wallet. In certain embodiments, for example, the de-identified (or anonymous or device-identified) data may be pushed to the distributed ledger from a peer in a distributed ledger network. In certain embodiments, for example, the pushing may comprise invoking a smart contract. In certain embodiments, for example, the pushing may comprise providing a quantity of a cryptocurrency.

In certain embodiments, for example, the network packet may be independently pushed from a mobile medical device. In certain embodiments, for example, the network packet may be pushed from the mobile medical device based on a scheduled prompt (for example a timed schedule). In certain embodiments, for example, the network packet may be pushed from the mobile medical device in response to detection of a communication network such as a cellular network, a Bluetooth connection, or Wi-Fi network. In certain embodiments, for example, the network packet may be pushed from the mobile medical device based on the size of the de-identified (or anonymous or device-identified) data reaching a threshold size in a memory of the mobile medical device.

In certain embodiments, for example, the pushing may comprise passing instructions (for example instructions comprising one or more JSON messages) and the second digital signature to a peer in a distributed ledger network, the instructions executable by the peer to add the hash to the distributed ledger. In certain embodiments, for example, the pushing may further comprise passing a digital signature for the HIPAA-compliant database applied at least to the instructions and the hash to the peer. In certain embodiments, for example, the digital signature for the HIPAA-compliant database may be applied to at least the instructions, the hash, and the second digital signature. In certain embodiments, for example, the instructions may be obtained from the second payload. In certain embodiments, for example, the instructions may be obtained from the second digital signature.

C. In certain embodiments, for example, the method may further comprise: authorizing data sources to form the authorized data sources, the authorizing comprising registering mobile medical devices. In certain embodiments, for example, the registering mobile medical devices (for example registering mobile medical devices via an online portal wherein a patient provides a registration code obtained from the mobile medical device) may comprise receiving executed HIPAA release forms from patients (for example receiving the HIPAA release forms digitally signed by the patients via an online registration portal). In certain embodiments, for example, the authorizing data sources may comprise associating the member of the list of public keys (which may also be a list of device identification codes or one may be derived from the other) with patient identification information (for example in the HIPAA-compliant database).

D. In certain embodiments, for example, the method may further comprise: authorizing access to at least one of the HIPAA-compliant database records, a block in a blockchain referencing the hash in the distributed ledger, and the hash in the distributed ledger. In certain embodiments, for example, the authorizing access may comprise a) forwarding an access request from a third party and b) receiving access permission for the third party from the patient. In certain embodiments, for example, the identity of the third party receiving access authorization may be stored in a specified distributed ledger. In certain embodiments, for example, the specified distributed ledger may be checked when the third party attempts to access the at least one of the HIPAA-compliant database records, the block in a blockchain referencing the hash in the distributed ledger, and the hash in the distributed ledger. In certain embodiments, for example, the third party attempt may comprise the third party providing a digitally signed request.

E. In certain embodiments, for example, the mobile medical devices may be attached to patients. In certain embodiments, for example, the mobile medical devices may be worn by patients. In certain embodiments, for example, the mobile medical devices may be ingested by patients. In certain embodiments, for example, the mobile medical devices may be injected into patients. In certain embodiments, for example, the mobile medical devices may be implanted into patients. In certain embodiments, for example, the mobile medical devices may gather and transmit data. In certain embodiments, for example, the mobile medical devices may be drug delivery devices. In certain embodiments, for example, the mobile medical devices may be coupled for data transmission with further mobile medical devices. In certain embodiments, for example, the mobile medical devices may gather and transmit data and the further mobile medical devices may be drug delivery devices. In certain embodiments, for example, the mobile medical devices may be drug delivery devices and the further mobile medical devices may gather and transmit data. In certain embodiments, for example, the mobile medical devices may be utilized inside a healthcare facility.

Certain embodiments may provide, for example, a method for processing data from mobile medical devices (for example a series of mobile devices produced by a single manufacturer). In certain embodiments, for example, the method may comprise receiving network packets, the network packets comprising first digital signatures and dual payloads, first payloads of the dual payloads comprising de-identified (or anonymous or device-identified) data, second payloads of the dual payloads comprising second digital signatures and hashes. In certain embodiments, for example, the method may comprise authenticating the network packets by verifying the first digital signatures using one or more signature verifying algorithms in combination with the first digital signatures, the dual payloads and members of a list of public keys assigned to authorized data sources (for example a cryptographic device identification key assigned to a mobile medical device) (for example recovering the dual payloads from at least the first digital signatures). In certain embodiments, for example, the method may comprise using the members of the list of public keys to identify patients. In certain embodiments, for example, the method may comprise inserting the de-identified (or anonymous or device-identified) data into records for the patients in at least one HIPAA-compliant database. In certain embodiments, for example, the method may comprise pushing the de-identified (or anonymous or device-identified) data to at least one distributed ledger without any patient identification information.

A. In certain embodiments, for example, the method may further comprise: passing at least a portion of the de-identified (or anonymous or device-identified) data through one or more computer models to obtain one or more data trends (for example one or more regressions). In certain embodiments, for example, the one or more computer models may comprise one or more statistical models. In certain embodiments, for example, the one or more computer models may comprise one or more neural networks. In certain embodiments, for example, the one or more data trends may comprise a regression (for example a linear, loglinear, logistic, or nonlinear regression). In certain embodiments, for example, the one or more data trends may comprise one or more parameters that indicate non-compliant use (for example may support a clinical determination of overuse or underuse) of the mobile medical device.

In certain embodiments, for example, the method may further comprise passing the one or more data trends to one or more healthcare agents. In certain embodiments, for example, the one or more healthcare agents may comprise one or more third-party reporting services. In certain embodiments, for example, the one or more healthcare agents may comprise one or more doctors. In certain embodiments, for example, the one or more healthcare agents may comprise one or more healthcare clinicians (for example one or more clinicians within the meaning of a CMS reimbursement code for reimbursing fee-for-service clinicians for monthly non-contact review of patient data for eligible patients). In certain embodiments, for example, the one or more healthcare agents may comprise one or more healthcare facilities. In certain embodiments, for example, the one or more healthcare agents may comprise one or more nurse practitioners.

Certain embodiments may provide, for example, a method for processing data from mobile medical devices. In certain embodiments, for example, the method may comprise receiving a network packet, the network packet comprising a first digital signature and a dual payload, a first payload of the dual payload comprising de-identified (or anonymous or device-identified) data, a second payload of the dual payload comprising a message, the message comprising a hash of the de-identified (or anonymous or device-identified) data, a second digital signature, and instructions (for example an embedded function call) for a peer of a distributed ledger network to add the hash to a distributed ledger. In certain embodiments, for example, the method may comprise authenticating the network packet by verifying the first digital signature using one or more signature verifying algorithms in combination with the first digital signature, the dual payload and a member of a list of public keys assigned to authorized data sources (for example a cryptographic device identification key assigned to a mobile medical device) (for example recovering the dual payload from at least the first digital signature). In certain embodiments, for example, the method may comprise using the member of the list of public keys to identify a patient. In certain embodiments, for example, the method may comprise inserting the de-identified (or anonymous or device-identified) data into a record for the patient, the record located in a HIPAA-compliant database. In certain embodiments, for example, the method may comprise pushing the message to the peer.

Certain embodiments may provide, for example, a method for processing data from mobile medical devices. In certain embodiments, for example, the method may comprise receiving network packets, the network packets comprising first digital signatures and dual payloads, first payloads of the dual payloads comprising de-identified (or anonymous or device-identified) data, second payloads of the dual payloads comprising messages, the messages comprising hashes of the de-identified (or anonymous or device-identified) data, second digital signatures, and instructions for at least one peer of at least one distributed ledger network to add the hashes to at least one distributed ledger. In certain embodiments, for example, the method may comprise authenticating the network packets by verifying the first digital signatures using one or more signature verifying algorithms in combination with the first digital signatures, the dual payloads and members of a list of public keys assigned to authorized data sources (for example a cryptographic device identification key assigned to a mobile medical device) (for example recovering the dual payloads from at least the first digital signatures). In certain embodiments, for example, the method may comprise using the members of the list of public keys to identify patients. In certain embodiments, for example, the method may comprise inserting the de-identified (or anonymous or device-identified) data into records for the patients in at least one HIPAA-compliant database. In certain embodiments, for example, the method may comprise pushing the messages to the at least one peer.

Certain embodiments may provide, for example, a method for processing data from mobile medical devices, comprising: i) receiving a network packet (for example via a communication pathway comprising a wireless network such as a cellular network or a Wi-Fi network), the network packet comprising a first digital signature and a dual payload, a first payload of the dual payload comprising de-identified (or anonymous or device-identified) data (for example timing and duration-of-use data from a mobile medical device such as a nebulizer issued to and registered to a patient), a second payload of the dual payload comprising a second digital signature and a hash; ii) authenticating the network packet by verifying the first digital signature using one or more signature verifying algorithms in combination with the first digital signature, the dual payload and a member of a list of public keys assigned to authorized data sources (for example a cryptographic device identification key assigned to a mobile medical device) (for example recovering the dual payload from at least the first digital signature); iii) using the member of the list of public keys to identify a patient (for example a patient who has been assigned a mobile medical device such as a patient under treatment for COPD who has been assigned a nebulizer); iv) inserting the de-identified (or anonymous or device-identified) data into a record for the patient in a HIPAA-compliant database (for example a HIPAA-compliant database maintained or curated by a manufacturer of a mobile medical device or a healthcare services business); and v) pushing the de-identified (or anonymous or device-identified) data to a distributed ledger (for example a public or private instance of a distributed ledger such as Ethereum) without any patient identification information.

Certain embodiments may provide, for example, a method for processing data from mobile medical devices (for example a series of mobile devices produced by a single manufacturer), comprising: i) receiving network packets, the network packets comprising first digital signatures and dual payloads, first payloads of the dual payloads comprising de-identified (or anonymous or device-identified) data, second payloads of the dual payloads comprising second digital signatures and hashes; ii) authenticating the network packets by verifying the first digital signatures using one or more signature verifying algorithms in combination with the first digital signatures, the dual payloads and members of a list of public keys assigned to authorized data sources (for example a cryptographic device identification key assigned to a mobile medical device) (for example recovering the dual payloads from at least the first digital signatures); iii) using the members of the list of public keys to identify patients; iv) inserting the de-identified (or anonymous or device-identified) data into records for the patients in at least one HIPAA-compliant database; and v) pushing the de-identified (or anonymous or device-identified) data to at least one distributed ledger without any patient identification information.

Certain embodiments may provide, for example, a method for processing data from mobile medical devices, comprising: i) receiving a network packet, the network packet comprising a first digital signature and a dual payload, a first payload of the dual payload comprising de-identified (or anonymous or device-identified) data, a second payload of the dual payload comprising a message, the message comprising a hash of the de-identified (or anonymous or device-identified) data, a second digital signature, and instructions for a peer of a distributed ledger network to add the hash to a distributed ledger; ii) authenticating the network packet by verifying the first digital signature using one or more signature verifying algorithms in combination with the first digital signature, the dual payload and a member of a list of public keys assigned to authorized data sources (for example a cryptographic device identification key assigned to a mobile medical device) (for example recovering the dual payload from at least the first digital signature); iii) using the member of the list of public keys to identify a patient; iv) inserting the de-identified (or anonymous or device-identified) data into a record for the patient, the record located in a HIPAA-compliant database; and v) pushing the message to the peer.

Certain embodiments may provide, for example, a method for processing data from mobile medical devices, comprising: i) receiving network packets, the network packets comprising first digital signatures and dual payloads, first payloads of the dual payloads comprising de-identified (or anonymous or device-identified) data, second payloads of the dual payloads comprising messages, the messages comprising hashes of the de-identified (or anonymous or device-identified) data, second digital signatures, and instructions for at least one peer of at least one distributed ledger network to add the hashes to at least one distributed ledger; ii) authenticating the network packets by verifying the first digital signatures using one or more signature verifying algorithms in combination with the first digital signatures, the dual payloads and members of a list of public keys assigned to authorized data sources (for example a cryptographic device identification key assigned to a mobile medical device) (for example recovering the dual payloads from at least the first digital signatures); iii) using the members of the list of public keys to identify patients; iv) inserting the de-identified (or anonymous or device-identified) data into records for the patients in at least one HIPAA-compliant database; and v) pushing the messages to the at least one peer.

Certain embodiments may provide, for example, a method for remote management of patient compliance. In certain embodiments, for example, the method may comprise issuing a mobile medical device (for example a hand-held nebulizer), with use instructions for treating a medical condition (for example medical condition that is classified as a program measure for computing a readmission charge against prior healthcare billings of the healthcare facility), to a patient, the medical device comprising: a processor, a memory in communication with the processor, a data source in communication with the processor, reference to predetermined network coordinates stored in the memory, and program code stored in the memory, the program code executable by the processor to perform data communication operations, the data communication operations comprising: a) processing signals from the data source to obtain device data; b) inserting dual payloads and device signatures applied to the dual payloads into network packets, first payloads of the dual payloads comprising at least portions of the device data, second payloads of the dual payloads comprising hashes of the at least portions and device signatures applied to the hashes; and c) pushing the network packets to the predetermined network coordinates in response to internally-generated prompts. In certain embodiments, for example, the method may comprise remotely monitoring (for example after a healthcare provider meets the patient in person (for example meeting regarding the medical condition)) at least one patient compliance parameter, comprising: a) downloading the at least portions of the device data from a HIPAA-compliant database; b) pulling the hashes and the device signatures applied to the hashes from a distributed ledger; c) authenticating the network packets, comprising verifying the digital signatures (for example recovering the dual payloads from the device signatures applied to the dual payloads); d) verifying the accuracies of the downloaded at least portions of the device data against the pulled hashes (for example passing the downloaded at least portions of the device data through a predetermined hash function to generate further hashes, and verifying expected matches between the further hashes and the pulled hashes); and e) processing the at least portions of the device data in combination with the use instructions to obtain the at least one patient compliance parameter (for example comparing the at least portions of the device data with acceptable values to compute a parameter indicating whether the patient has deviated from an acceptable course of treatment, the acceptable values computed based on the use instructions).

A. In certain embodiments, for example, the mobile medical device may have an assigned device activation code, the activation code interpretable by a web portal to assign the mobile medical device to a patient in the HIPAA-compliant database. In certain embodiments, for example, the activation code may be further interpretable by a web portal to permission the HIPAA-compliant database to receive the at least portions of the device data. In certain embodiments, for example, the assigned device activation code may be affixed to the mobile medical device. In certain embodiments, for example, the assigned device activation code may be affixed as scannable bar code on an outer surface of the mobile medical device. In certain embodiments, for example, the assigned device activation code may be affixed to the mobile medical device as a removable sticker.

B. In certain embodiments, for example, the mobile medical device may have an assigned access code, the access code interpretable by a web portal to request permission from the patient to provide access to the at least portions of the device data in the HIPAA-compliant database to a registered party. In certain embodiments, for example, the access code may be affixed to the mobile medical device. In certain embodiments, for example, the access code may be affixed as scannable bar code on an outer surface of the mobile medical device. In certain embodiments, for example, a public key for verifying the device signatures (for example by recovering the dual payloads from the device signatures applied to the dual payloads) may be issued to a registered party upon approval of the requested permission. In certain embodiments, for example, access permission may be revoked by the patient.

C. In certain embodiments, for example, the data source may comprise a sensor. In certain embodiments, for example, the data source may comprise a dispensing element. In certain embodiments, for example, the data source may comprise an electronic circuit. In certain embodiments, for example, the data source may comprise a data aggregation unit. In certain embodiments, for example, the data source may comprise an actuator.

D. In certain embodiments, for example, the mobile medical device may further comprise a mechanical actuator, wherein the data source is activated into a data collection mode by the mechanical actuator. In certain embodiments, for example, the mechanical actuator may be configured for operation by the patient (for example the mechanical actuator may be a button. In certain embodiments, for example, the data source actuated by the mechanical actuator may comprise an electronic circuit.

E. In certain embodiments, for example, the mobile medical device may be issued to or prescribed to the patient during a visit to a healthcare facility. In certain embodiments, for example, the visit may comprise hospitalization. In certain embodiments, for example, the visit may comprise an outpatient visit. In certain embodiments, for example, the method may reduce the probability of readmission of the patient to the healthcare facility or to a different healthcare for treatment of the medical condition for by at least 5%, for example at least 10%, at least 15%, at least 20%, at least 25%, at least 30%, or the method may reduce the probability of readmission of the patient to the healthcare facility or to a different healthcare for treatment of the medical condition for by at least 40%. In certain embodiments, for example, the method may reduce the probability of readmission of the patient to the healthcare facility or to a different healthcare for treatment of the medical condition for by at least 5%, for example at least 10%, at least 15%, at least 20%, at least 25%, at least 30%, or the method may reduce the probability of readmission of the patient to the healthcare facility or to a different healthcare for treatment of the medical condition for by at least 40% for at least 5 days (for example at least 10 days, at least 20 days, at least 30 days, or at least 30 days) following departure of the patient from the healthcare facility. In certain embodiments, for example, the method may reduce the probability of readmission of the patient to the healthcare facility or to a different healthcare for treatment of the medical condition for by at least 30% for 30 days following departure of the patient from the healthcare facility. In certain embodiments, for example, the mobile medical device may be issued prior to the patient leaving the healthcare facility. In certain embodiments, for example, the mobile medical device may be obtained for issue from an inventory of the healthcare facility. In certain embodiments, for example, the mobile medical device may further comprise a wireless interface (for example a cellular radio). In certain embodiments, for example, the mobile medical device may be a nebulizer. In certain embodiments, for example, the mobile medical device may be a vibrating mesh nebulizer. In certain embodiments, for example, the mobile medical device may be a handheld nebulizer. In certain embodiments, for example, the mobile medical device may be an inhaler.

F. In certain embodiments, for example, the use instructions may be obtained from (or derived from) a medical prescription for the patient. In certain embodiments, for example, the use instructions may be obtained from a product label for a medication. In certain embodiments, for example, the use instructions may be obtained from an electronic health records database. In certain embodiments, for example, the electronic health records database may be separate from the HIPAA-compliant database and the distributed ledger.

G. In certain embodiments, for example, the medical condition may comprise one or more of acute myocardial infarction (AMI), heart failure (HF), pneumonia (PN), chronic obstructive pulmonary disease (COPD), asthma, elective hip arthroplasty (THA), total knee arthroplasty (TKA), stroke, diabetes (DM), hypertension (HTN), dysrhythmia, and obstructive sleep apnea (OSA). In certain embodiments, for example, the medical condition may be classified as a program measure for computing a readmission charge against prior healthcare billings of the healthcare facility. In certain embodiments, for example, the program measure may apply to public insurance. In certain embodiments, for example, the patient may be dually eligible for Medicare and full-benefit Medicaid. In certain embodiments, for example, the program measure may apply to private insurance.

H. In certain embodiments, for example, the device data may be owned by the patient. In certain embodiments, for example, the method may preserve ownership of the data by the patient. In certain embodiments, for example, the device data may comprise one or more of time-of-use, frequency of use, and use-duration data for the mobile medical device. In certain embodiments, for example, the device data may be non-biological data.

I. In certain embodiments, for example, the dual payloads may be recovered from the device signatures applied to the dual payloads using a public key. In certain embodiments, for example, the public key may be created by a manufacturer of the mobile medical device. In certain embodiments, for example, the second payloads may contain no patient identification information (for example no names, social security numbers, or healthcare provider-generated patient identifiers). In certain embodiments, for example, the second payloads may be de-identified (for example de-identified within the meaning prescribed by HIPAA). In certain embodiments, for example, the second payloads may comprise one or more instructions for inserting the hashes into the distributed ledger. In certain embodiments, for example, the one or more instructions may be recovered from the device signatures applied to the hashes.

J. In certain embodiments, for example, the distributed ledger may be a public distributed ledger. In certain embodiments, for example, the distributed ledger may be a private distributed ledger. In certain embodiments, for example, the distributed ledger may be a private distributed ledger configured to write (for example push) verification information (for example a hash of data added to the private distributed ledger) to a public ledger.

K. In certain embodiments, for example, the internally-generated prompts may comprise date-driven prompts. In certain embodiments, for example, the internally-generated prompts may be generated in response to detection of one or more wireless networks (for example one or more cellular networks).

L. In certain embodiments, for example, the data communication operations may further comprise: negotiating a dedicated network connection for pushing the network packets. In certain embodiments, for example, the dedicated network connection may be encrypted (for example the dedicated network connection may be an SSL connection or a TLS connection). In certain embodiments, for example, the dedicated network connection may utilize a TCP protocol. In certain embodiments, for example, the dedicated network connection may utilize a UDP protocol.

M. In certain embodiments, for example, the data communication operations may further comprise: locking down the mobile medical device to prevent incoming network communications from altering an application space of the memory. In certain embodiments, for example, the data communication operations may comprise dropping at least a portion (for example all) of incoming network packets in a kernel space of the mobile medical device. In certain embodiments, for example, the data communication operations may restrict open software ports to a single port having a preconfigured port number. In certain embodiments, for example, the data communication operations may restrict network connections to a connection between a local port having a preconfigured port number and singular destination coordinates. In certain embodiments, for example, the data communication operations may close any ports opened by non-preauthorized data communication operations. In certain embodiments, for example, the data communication operations may close any ports which have a port number different from a preconfigured port number. In certain embodiments, for example, the mobile medical device further comprises a network security agent. In certain embodiments, for example, the mobile medical device further comprises a firewall. In certain embodiments, for example, the mobile medical device further comprises anti-virus software. In certain embodiments, for example, the data communication operations may comprise forming a virtual private network connection.

N. In certain embodiments, for example, the remotely monitoring may be triggered by issuance of the mobile medical device. In certain embodiments, for example, the remotely monitoring may be triggered by registration of the mobile medical device. In certain embodiments, for example, the remotely monitoring may be triggered directly or indirectly by the pushing the network packets to the predetermined network coordinates (for example following registration of the mobile medical device to a patient). In certain embodiments, for example, the remotely monitoring may be performed periodically (for example repeated daily, weekly, monthly, and/or annually). In certain embodiments, for example, the remotely monitoring may be performed by a healthcare provider for at least 30 minutes in a month (or for at least 30 minutes each for at least two months, or for at least 30 minutes each in at least two consecutive months). In certain embodiments, for example, the healthcare provider may meet the patient in person prior to the reducing. In certain embodiments, for example, the remotely monitoring may further comprise: pulling digital signatures for the HIPAA-compliant database applied to the hashes. In certain embodiments, for example, at least a portion of the remotely monitoring may be performed by at least one reporting service (for example a third-party reporting service).

O. In certain embodiments, for example, the at least one patient compliance parameter may comprise a measure of patient compliance with the use instructions (for example a comparison of use instructions with at least one of device usage frequency and device usage duration). In certain embodiments, for example, the at least one patient compliance parameter may comprise a measure of patient non-compliance with the use instructions. In certain embodiments, for example, the measure of patient non-compliance may comprise a cumulative number of missed treatments by the mobile medical device. In certain embodiments, for example, the measure of patient non-compliance may comprise a cumulative quantity of medication. In certain embodiments, for example, the at least one patient compliance parameter may comprise a probability of readmission of the patient to a healthcare facility to treat the medical condition. In certain embodiments, for example, the at least one patient compliance parameter may comprise an estimate of an excess readmission rate.

P. In certain embodiments, for example, the downloading may comprise at least 1 download (for example at least 2 downloads, at least 3 downloads, at least 4 downloads, at least 5 downloads, at least 6 downloads, at least 7 downloads, at least 8 downloads, at least 9 downloads, at least 10 downloads, at least 11 downloads, at least 12 downloads, at least 13 downloads, at least 14 downloads, at least 15 downloads, at least 16 downloads, at least 17 downloads, at least 18 downloads, at least 19 downloads, at least 20 downloads, at least 21 downloads, at least 22 downloads, at least 23 downloads, at least 24 downloads, or at least 25 downloads), the at least 1 download performed between 0 (for example 1) and 25 days (for example daily downloads for at least 10 days, 15 days, or at least 20 days following the patient leaving (for example being discharged from) a healthcare facility. In certain embodiments, for example, at least one of the downloading and the pulling may utilize the public Internet. In certain embodiments, for example, at least one of the downloading and the pulling may utilize a virtual private network. In certain embodiments, for example, at least one of the downloading and the pulling may utilize a peer in a distributed ledger network. In certain embodiments, for example, at least one of the downloading and the pulling may utilize a wallet, the wallet configured for communication with a distributed ledger network. In certain embodiments, for example, the HIPAA-compliant database may be a portion of the distributed ledger or a further distributed ledger. In certain embodiments, for example, at least one of the HIPAA-compliant database and the distributed ledger may be maintained in cloud-computing resource.

Q. In certain embodiments, for example, the hashes and the device signatures may be pulled via a dedicated peer in a distributed ledger network hosting the distributed ledger. In certain embodiments, for example, pulling the hashes and the device signatures may comprise executing at least one smart contract on the distributed ledger network. In certain embodiments, for example, an input to the at least one smart contract may comprise a quantity of a cryptocurrency. In certain embodiments, for example, the cryptocurrency may be a quantity of a bitcoin. In certain embodiments, for example, the cryptocurrency may have been issued by an initial coin offering. In certain embodiments, for example, the cryptocurrency may be a gas used to fund execution of one or more smart contracts and/or a transaction confirmation process.

R. In certain embodiments, for example, the verifying may comprise obtaining the hashes by passing the downloaded at least portions of the device data through a hash function.

S. In certain embodiments, for example, the processing the at least portions of the device data in combination with the use instructions may comprise using the at least portions of the device data to estimate consumption of a medication by the patient. In certain embodiments, for example, the processing may utilize disease information for the patient. In certain embodiments, for example, the processing utilizes one or more of prior admittance and facility type information for the patient. In certain embodiments, for example, the processing may utilize demographic information for the patient.

T. In certain embodiments, for example, the processing may comprise inputting the at least portions of the device data and the use instructions into a predictive model to determine the at least one patient compliance parameter, the predictive model configured with risk variables and risk thresholds of the medical condition. In certain embodiments, for example, the at least one patient compliance parameter may comprise a risk of readmission of the patient. In certain embodiments, for example, the predictive model may generate an estimate of an excess readmission rate (for example an excess readmission rate within the meaning specified by a Hospital Readmissions Reduction Program (HRRP) administered by the CMS). In certain embodiments, for example, the predictive model may depend on a cumulative number of patients treated for the medical condition. In certain embodiments, for example, the predictive model may depend on a cumulative number of patients treated for one or more further medical conditions. In certain embodiments, for example, the remotely monitoring may comprise: adjusting a schedule for one or more of the pulling, downloading, and processing if the determined risk of readmission exceeds a first threshold. In certain embodiments, for example, the remotely monitoring may further comprise: triggering an intervention protocol if the determined risk of readmission exceeds a second threshold. In certain embodiments, for example, the remotely monitoring may further comprise: updating the predictive model based on whether the patient is admitted to a healthcare facility for treatment of the medical condition within 30 days of the patient leaving (for example being discharged from) a healthcare facility. In certain embodiments, for example, the method may further comprise: triggering an intervention protocol if a) the determined risk of readmission exceeds a pre-determined threshold and b) readmission of the patient within 30 days would increase a readmission charge by more than a predetermined amount. In certain embodiments, for example, the method may further comprise: triggering an intervention protocol if a) the determined risk of readmission exceeds a pre-determined threshold and b) readmission of the patient within 30 days would increase an excess readmission ratio by more than a predetermined amount. In certain embodiments, for example, the at least one patient compliance parameter may be an economic risk of readmission, the predictive model configured with risk variables and risk thresholds of a plurality of program measures for computing a readmission charge. In certain embodiments, for example, the remotely monitoring may further comprise: triggering an intervention protocol if the determined economic risk of readmission exceeds a pre-determined threshold.

U. In certain embodiments, for example, the remotely monitoring may further comprise: authenticating the hashes, comprising verifying the digital signatures applied to the hashes (for example by recovering the hashes from the device signatures applied to the hashes). In certain embodiments, for example, the remotely monitoring may further comprise: requesting access to the at least portions of the device data. In certain embodiments, for example, the remotely monitoring may further comprise: providing a computer interface for the patient to register the mobile medical device. In certain embodiments, for example, the remotely monitoring may further comprise: using the at least one patient compliance parameter to determine a level of patient compliance with the use instructions. In certain embodiments, for example, the remotely monitoring may further comprise: contacting the patient if the level of patient compliance is below a predetermined level.

Certain embodiments may provide, for example, a method for remote management of patient compliance. In certain embodiments, for example, the method may comprise issuing mobile medical devices, with use instructions for treating medical conditions, to patients, the medical devices comprising: processors, memories in communication with the processors, data sources in communication with the processors, predetermined network coordinates stored in the memories, and program codes stored in the memories, the program codes executable by the processors to perform data communication operations, the data communication operations comprising: a) processing signals from the data sources to obtain device data; b) inserting dual payloads and device signatures applied to the dual payloads into network packets, first payloads of the dual payloads comprising at least portions of the device data, second payloads of the dual payloads comprising hashes of the at least portions and device signatures applied to the hashes; and c) pushing the network packets to the predetermined network coordinates in response to internally-generated prompts. In certain embodiments, for example, the method may comprise remotely monitoring at least one patient compliance parameter, comprising: a) downloading the at least portions of the device data from at least one HIPAA-compliant database; b) pulling the hashes and the device signatures applied to the hashes from at least one distributed ledger; c) authenticating the network packets by verifying the device signatures (for example by recovering the dual payloads from the device signatures applied to the dual payloads); d) verifying the accuracies of the downloaded at least portions of the device data against the pulled hashes; and e) processing the at least portions of the device data in combination with the use instructions to obtain the at least one patient compliance parameter.

A. In certain embodiments, for example, the medical conditions may comprise one or more of acute myocardial infarction (AMI), heart failure (HF), pneumonia (PN), chronic obstructive pulmonary disease (COPD), asthma, elective hip arthroplasty (THA), total knee arthroplasty (TKA), stroke, diabetes (DM), hypertension (HTN), dysrhythmia, and obstructive sleep apnea (OSA). In certain embodiments, for example, at least a portion of the patients may be dually eligible for Medicare and full-benefit Medicaid.

B. In certain embodiments, for example, at least a portion of the remotely monitoring may be performed at one or more of the healthcare facilities. In certain embodiments, for example, at least a portion of the remotely monitoring may be performed by at least one reporting service (for example at least one reporting service utilizing an application programming interface).

C. In certain embodiments, for example, the at least one patient compliance parameter may comprise at least one readmission rate for at least one of the medical conditions. In certain embodiments, for example, the at least one patient compliance parameter may comprise at least one patient compliance measure for at least one prescribed course of treatment. In certain embodiments, for example, the at least one patient compliance parameter may be a trailing rate of readmission of at least a portion of the patients. In certain embodiments, for example, the method may reduce the trailing rate of readmission by at least 5% (for example at least 10%, at least 15%, at least 20%, at least 25%, at least 30%, at least 40%, at least 50%, or the method may reduce the trailing rate of readmission by at least 70%. In certain embodiments, for example, the trailing rate of readmission may be an at least a 30-day (or at least a 10-day, at least a 15-day, at least a 45-day, at least a 2-month, at least a 3-month, at least a 4-month, at least a 6-month, at least a 8-month, at least a 1-year, or at least a 2-year) trailing rate applied to patients who have left healthcare facilities in the past 30 days (or within 10 days, or within 15 days, or within 20 days, or within 45 days) (for example leaving healthcare facilities where treatment for one or more of the medical conditions was rendered).

D. In certain embodiments, for example, the processing may comprise inputting the at least portions of the device data and the use instructions into a predictive model (for example a predictive model derived by machine learning) to determine the at least one patient compliance parameter, the predictive model configured with risk variables and risk thresholds of the medical condition. In certain embodiments, for example, the processing may comprise providing the at least portions of the device data and use instructions to a machine learning platform (for example a machine learning platform comprising program code implementing one or more machine learning algorithms). In certain embodiments, for example, the processing may comprise inputting the at least portions of the device data and the use instructions into a classification model to determine the at least one patient compliance state, treatment state, and/or disease state. In certain embodiments, for example, the processing may comprise computing, using the predictive model, risk scores in response to the risk variables and risk thresholds that is indicative of the patients' risk for developing the medical conditions. In certain embodiments, for example, the processing may further comprise, in response to the computed risk scores, identifying at least one high-risk patient as having a high risk of readmission to a healthcare facility for treatment of at least one of the medical conditions. In certain embodiments, for example, the method may further comprise: presenting at least one of visual and audible notification to an intervention coordination team about the identified at least one high-risk patient. In certain embodiments, for example, the method may further comprise: presenting at least one of visual and audible notification to the identified at least one high-risk patient. In any of the foregoing embodiments, for example, at least one of visual and audible notification may be provided by an automated intervention system.

E. In certain embodiments, for example, the remotely monitoring may further comprise: authenticating the hashes, comprising verifying the device signatures applied to the hashes (for example by recovering the hashes from the device signatures applied to the hashes).

Certain embodiments may provide, for example, a method for remote management of patient compliance (for example remote patient compliance qualifying for at least partial reimbursement by CMS, such as reimbursing fee-for-service clinicians for monthly non-contact review of patient data for eligible patients). In certain embodiments, for example, the method may comprise issuing a mobile medical device, with use instructions for treating a medical condition, to a patient, the medical device comprising: a processor, a memory in communication with the processor, a data source in communication with the processor, reference to predetermined network coordinates stored in the memory, and program code stored in the memory, the program code executable by the processor to perform data communication operations, the data communication operations comprising: a) processing signals from the data source to obtain device data; b) inserting a dual payload and a device signature applied to the dual payload into a network packet, a first payload of the dual payload comprising at least a portion of the device data, a second payload of the dual payload comprising a hash of the at least a portion and a device signature applied to the hash; and c) pushing the network packet to the predetermined network coordinates in response to an internally-generated prompt. In certain embodiments, for example, the method may comprise remotely monitoring at least one patient compliance parameter (for example, remotely monitoring by a clinician at least 30 minutes per month), comprising: a) downloading the at least a portion of the device data from a HIPAA-compliant database; b) pulling the hash and the device signature applied to the hash from a distributed ledger; c) authenticating the hash by verifying the digital signatures applied to the hash (for example by recovering the hash from at least the device signature applied to the hash); d) verifying the accuracy of the downloaded at least a portion of the device data against the pulled hash; and e) processing the at least a portion of the device data in combination with the use instructions to obtain the at least one patient compliance parameter.

A. In certain embodiments, for example, the dual payloads may be recovered from the device signature applied to the dual payload using a public key. In certain embodiments, for example, the public key (and/or a device identification code) may be created by a manufacturer of the mobile medical device. In certain embodiments, for example, the second payload may contain no patient identification information. In certain embodiments, for example, the second payload may be de-identified. In certain embodiments, for example, the second payload may comprise one or more instructions for inserting the hash into the distributed ledger. In certain embodiments, for example, the one or more instructions may be recovered from the device signature applied to the hash.

B. In certain embodiments, for example, the internally-generated prompt may comprise a date-driven prompt. In certain embodiments, for example, the internally-generated prompt may be generated in response to detection of one or more wireless networks. In certain embodiments, for example, the internally-generated prompt may be triggered by a positive comparison of device-generated data and one or more predetermined values (for example critical usage or safety parameters). In certain embodiments, for example, the internally-generated prompt may be triggered when a portion of the device data stored in the memory exceeds a predetermined value.

C. In certain embodiments, for example, the data communication operations may further comprise: negotiating a dedicated network connection for pushing the network packet.

D. In certain embodiments, for example, the remotely monitoring may further comprise: pulling a digital signature for the HIPAA-compliant database applied to the hash.

E. In certain embodiments, for example, the downloading may comprise a download performed between 1 and 25 days following the patient leaving a healthcare facility (for example leaving a healthcare following treatment at least for the medical condition).

F. In certain embodiments, for example, the hash and the device signature may be pulled via a dedicated peer in a distributed ledger network hosting the distributed ledger. In certain embodiments, for example, pulling the hash and the device signature may comprise executing at least one smart contract on the distributed ledger network.

G. In certain embodiments, for example, the verifying may comprise obtaining the hash by passing the downloaded at least a portion of the device data through a hash function.

H. In certain embodiments, for example, the processing the at least a portion of the device data in combination with the use instructions may comprise using the at least a portion of the device data to estimate consumption of a medication by the patient.

I. In certain embodiments, for example, the processing may comprise inputting the at least a portion of the device data and the use instructions into a predictive model to determine the at least one patient compliance parameter, the predictive model configured with risk variables and risk thresholds of the medical condition.

J. In certain embodiments, for example, the remotely monitoring may further comprise: authenticating the hash, comprising verifying the device signatures applied to the hash (for example by recovering the hash from the device signatures applied to the hash). In certain embodiments, for example, the remotely monitoring may further comprise: requesting access to the at least a portion of the device data.

Certain embodiments may provide, for example, a method for remote management of patient compliance. In certain embodiments, for example, the method may comprise issuing a mobile medical device, with use instructions for treating a medical condition, to a patient, the medical device comprising: a processor, a memory in communication with the processor, a data source in communication with the processor, reference to predetermined network coordinates stored in the memory, and program code stored in the memory, the program code executable by the processor to perform data communication operations, the data communication operations comprising: a) processing signals from the data source to obtain device data; b) inserting a dual payload and a device signature applied to the dual payload into a network packet, a first payload of the dual payload comprising at least a portion of the device data, a second payload of the dual payload comprising a message, the message comprising a hash of the at least a portion, a device signature applied to the hash, and instructions for a peer of a distributed ledger network to add the hash to a distributed ledger; and c) pushing the network packet to the predetermined network coordinates in response to an internally-generated prompt. In certain embodiments, for example, the method may comprise remotely monitoring at least one patient compliance parameter, comprising: a) downloading the at least a portion of the device data from a HIPAA-compliant database; b) pulling the hash and the device signature applied to the hash from the distributed ledger; c) authenticating the hash by verifying the device signature applied to the hash (for example by recovering the hash from at least the device signature applied to the hash); d) verifying the accuracy of the downloaded at least a portion of the device data against the pulled hash; and e) processing the at least a portion of the device data in combination with the use instructions to obtain the at least one patient compliance parameter.

A. In certain embodiments, for example, the dual payload may be recovered from at least a digital signature for the HIPAA-compliant database applied to the device signature applied to the dual payload.

Certain embodiments may provide, for example, a method to reduce readmission rates at healthcare facilities. In certain embodiments, for example, the method may comprise issuing mobile medical devices, with use instructions for treating medical conditions, to patients who have visited healthcare facilities, the medical devices comprising: processors, memories in communication with the processors, data sources in communication with the processors, reference to predetermined network coordinates stored in the memories, and program codes stored in the memories, the program codes executable by the processors to perform data communication operations, the data communication operations comprising: a) processing signals from the data sources to obtain device data; b) inserting dual payloads and device signatures applied to the dual payloads into network packets, first payloads of the dual payloads comprising at least portions of the device data, second payloads of the dual payloads comprising hashes of the at least portions and device signatures applied to the hashes; and c) pushing the network packets to the predetermined network coordinates in response to internally-generated prompts. In certain embodiments, for example, the method may comprise reducing at least one readmission rate at the healthcare facilities for at least one of the medical conditions, comprising: a) downloading the at least portions of the device data from at least one HIPAA-compliant database; b) pulling the hashes and the device signatures applied to the hashes from at least one distributed ledger; c) authenticating the network packets by verifying the digital signatures applied to the dual payloads (for example by recovering the dual payloads from the device signatures applied to the dual payloads); d) verifying the accuracies of the downloaded at least portions of the device data against the pulled hashes; and e) processing the at least portions of the device data in combination with the use instructions to determine risks of readmission of the patients.

Certain embodiments may provide, for example, a method to reduce a readmission rate at a healthcare facility. In certain embodiments, for example, the method may comprise issuing a mobile medical device, with use instructions for treating a medical condition, to a patient who has visited a healthcare facility, the medical device comprising: a processor, a memory in communication with the processor, a data source in communication with the processor, reference to predetermined network coordinates stored in the memory, and program code stored in the memory, the program code executable by the processor to perform data communication operations, the data communication operations comprising: a) processing signals from the data source to obtain device data; b) inserting dual payloads and device signatures applied to the dual payloads into network packets, first payloads of the dual payloads comprising at least portions of the device data, second payloads of the dual payloads comprising hashes of the at least portions and device signatures applied to the hashes; and c) pushing the network packets to the predetermined network coordinates in response to internally-generated prompts. In certain embodiments, for example, the method may comprise reducing a readmission rate at the healthcare facility for the medical condition, comprising: a) downloading the at least portions of the device data from a HIPAA-compliant database; b) pulling the hashes and the device signatures applied to the hashes from a distributed ledger; c) authenticating the network packets, comprising verifying the device signatures applied to the dual payloads (for example by recovering the dual payloads from the device signatures applied to the dual payloads); d) verifying the accuracies of the downloaded at least portions of the device data against the pulled hashes; and e) processing the at least portions of the device data in combination with the use instructions to determine risks of readmission of the patient.

Certain embodiments may provide, for example, a method to reduce a readmission rate at a healthcare facility. In certain embodiments, for example, the method may comprise issuing a mobile medical device, with use instructions for treating a medical condition, to a patient who has visited a healthcare facility, the medical device comprising: a processor, a memory in communication with the processor, a data source in communication with the processor, reference to predetermined network coordinates stored in the memory, and program code stored in the memory, the program code executable by the processor to perform data communication operations, the data communication operations comprising: a) processing signals from the data source to obtain device data; b) inserting a dual payload and a device signature applied to the dual payload into a network packet, a first payload of the dual payload comprising at least a portion of the device data, a second payload of the dual payload comprising a hash of the at least a portion and a device signature applied to the hash; and c) pushing the network packet to the predetermined network coordinates in response to an internally-generated prompt. In certain embodiments, for example, the method may comprise reducing a readmission rate at the healthcare facility for the medical condition, comprising: a) downloading the at least a portion of the device data from a HIPAA-compliant database; b) pulling the hash and the device signature applied to the hash from a distributed ledger; c) authenticating the hash by verifying the device signature applied to the hash (for example by recovering the hash from at least the device signature applied to the hash); d) verifying the accuracy of the downloaded at least a portion of the device data against the pulled hash; and e) processing the at least a portion of the device data in combination with the use instructions to determine a risk of readmission of the patient.

Certain embodiments may provide, for example, a method for a healthcare provider to improve remote patient compliance monitoring of use of mobile medical devices by patients. In certain embodiments, for example, the method may comprise downloading at least portions of timing and duration-of-use data for the mobile medical devices from at least one HIPAA-compliant database. In certain embodiments, for example, the method may comprise pulling hashes and first digital signatures from at least one distributed ledger. In certain embodiments, for example, the method may comprise authenticating the hashes, comprising verifying the first digital signatures applied to the hashes using public keys assigned to the mobile medical devices (for example by recovering the hashes from the first digital signatures using the public keys assigned to the mobile medical devices). In certain embodiments, for example, the method may comprise verifying the accuracy of the at least portions of downloaded timing and duration-of-use data against the pulled hashes. In certain embodiments, for example, the method may comprise processing the at least portions of downloaded timing and duration-of-use data in combination with use instructions for the mobile medical devices to detect non-compliant uses of the mobile medical devices. In certain embodiments, for example, the method may comprise assigning a level of risk associated with diagnosed health conditions of the patients due to the non-compliant uses. In certain embodiments, for example, the method may comprise triggering an intervention protocol if the level of risk exceeds a pre-determined threshold.

Certain embodiments may provide, for example, a method for a healthcare provider to improve remote patient compliance monitoring of use of a mobile medical device by a patient. In certain embodiments, for example, the method may comprise downloading timing and duration-of-use data for the mobile medical device from a HIPAA-compliant database. In certain embodiments, for example, the method may comprise pulling at least one hash and at least a first digital signature from a distributed ledger. In certain embodiments, for example, the method may comprise authenticating the at least one hash, comprising verifying the at least a first digital signature applied to the hash using a public key assigned to the mobile medical device (for example by recovering the hash from the at least a first digital signature using the public key assigned to the mobile medical device). In certain embodiments, for example, the method may comprise verifying the accuracy of the downloaded timing and duration-of-use data against the pulled at least one hash. In certain embodiments, for example, the method may comprise processing the downloaded timing and duration-of-use data in combination with use instructions for the mobile medical device to detect non-compliant use (for example underuse or overuse) of the mobile medical device. In certain embodiments, for example, the method may comprise assigning a level of risk associated with at least one diagnosed health condition of a patient due to the uninstructed use. In certain embodiments, for example, the method may comprise triggering an intervention protocol if the level of risk exceeds a pre-determined threshold.

A. In certain embodiments, for example, the method may further comprise: conducting an in-person consultation with the patient regarding a medical condition, the medical device utilized in a course of treatment of the medical condition. In certain embodiments, for example, the downloading may comprise at least monthly downloads of portions of the timing and duration-of-use data.

Certain embodiments may provide, for example, a method for remote management of patient compliance, comprising: i) issuing a mobile medical device (for example a hand-held nebulizer), with use instructions for treating a medical condition (for example medical condition that is classified as a program measure for computing a readmission charge against prior healthcare billings of the healthcare facility), to a patient, the medical device comprising: a processor, a memory in communication with the processor, a data source in communication with the processor, reference to predetermined network coordinates stored in the memory, and program code stored in the memory, the program code executable by the processor to perform data communication operations, the data communication operations comprising: a) processing signals from the data source to obtain device data; b) inserting dual payloads and device signatures applied to the dual payloads into network packets, first payloads of the dual payloads comprising at least portions of the device data, second payloads of the dual payloads comprising hashes of the at least portions and device signatures applied to the hashes; and c) pushing the network packets to the predetermined network coordinates in response to internally-generated prompts; and ii) remotely monitoring (for example after a healthcare provider meets the patient in person (for example meeting regarding the medical condition)) at least one patient compliance parameter, comprising: a) downloading the at least portions of the device data from a HIPAA-compliant database; b) pulling the hashes and the device signatures applied to the hashes from a distributed ledger; c) authenticating the network packets, comprising verifying the device signatures applied to the dual payloads (for example by recovering the dual payloads from the device signatures applied to the dual payloads); d) verifying the accuracies of the downloaded at least portions of the device data against the pulled hashes (for example passing the downloaded at least portions of the device data through a predetermined hash function to generate further hashes, and verifying expected matches between the further hashes and the pulled hashes); and e) processing the at least portions of the device data in combination with the use instructions to obtain the at least one patient compliance parameter (for example comparing the at least portions of the device data with acceptable values to compute a parameter indicating whether the patient has deviated from an acceptable course of treatment, the acceptable values computed based on the use instructions).

Certain embodiments may provide, for example, a method for remote management of patient compliance, comprising: i) issuing mobile medical devices, with use instructions for treating medical conditions, to patients, the medical devices comprising: processors, memories in communication with the processors, data sources in communication with the processors, predetermined network coordinates stored in the memories, and program codes stored in the memories, the program codes executable by the processors to perform data communication operations, the data communication operations comprising: a) processing signals from the data sources to obtain device data; b) inserting dual payloads and device signatures applied to the dual payloads into network packets, first payloads of the dual payloads comprising at least portions of the device data, second payloads of the dual payloads comprising hashes of the at least portions and device signatures applied to the hashes; and c) pushing the network packets to the predetermined network coordinates in response to internally-generated prompts; and ii) remotely monitoring at least one patient compliance parameter, comprising: a) downloading the at least portions of the device data from at least one HIPAA-compliant database; b) pulling the hashes and the device signatures applied to the hashes from at least one distributed ledger; c) authenticating the network packets by verifying the device signatures applied to the dual payloads (for example by recovering the dual payloads from the device signatures applied to the dual payloads); d) verifying the accuracies of the downloaded at least portions of the device data against the pulled hashes; and e) processing the at least portions of the device data in combination with the use instructions to obtain the at least one patient compliance parameter.

Certain embodiments may provide, for example, a method for remote management of patient compliance (for example remote patient compliance qualifying for at least partial reimbursement by CMS, such as reimbursing fee-for-service clinicians for monthly non-contact review of patient data for eligible patients), comprising: i) issuing a mobile medical device, with use instructions for treating a medical condition, to a patient, the medical device comprising: a processor, a memory in communication with the processor, a data source in communication with the processor, reference to predetermined network coordinates stored in the memory, and program code stored in the memory, the program code executable by the processor to perform data communication operations, the data communication operations comprising: a) processing signals from the data source to obtain device data; b) inserting a dual payload and a device signature applied to the dual payload into a network packet, a first payload of the dual payload comprising at least a portion of the device data, a second payload of the dual payload comprising a hash of the at least a portion and a device signature applied to the hash; and c) pushing the network packet to the predetermined network coordinates in response to an internally-generated prompt; and ii) remotely monitoring at least one patient compliance parameter (for example, remotely monitoring by a clinician at least 30 minutes per month), comprising: a) downloading the at least a portion of the device data from a HIPAA-compliant database; b) pulling the hash and the device signature applied to the hash from a distributed ledger; c) authenticating the hash by verifying the device signature applied to the hash (for example by recovering the hash from at least the device signature applied to the hash); d) verifying the accuracy of the downloaded at least a portion of the device data against the pulled hash; and e) processing the at least a portion of the device data in combination with the use instructions to obtain the at least one patient compliance parameter.

Certain embodiments may provide, for example, a method for remote management of patient compliance, comprising: i) issuing a mobile medical device, with use instructions for treating a medical condition, to a patient, the medical device comprising: a processor, a memory in communication with the processor, a data source in communication with the processor, reference to predetermined network coordinates stored in the memory, and program code stored in the memory, the program code executable by the processor to perform data communication operations, the data communication operations comprising: a) processing signals from the data source to obtain device data; b) inserting a dual payload and a device signature applied to the dual payload into a network packet, a first payload of the dual payload comprising at least a portion of the device data, a second payload of the dual payload comprising a message, the message comprising a hash of the at least a portion, a device signature applied to the hash, and instructions for a peer of a distributed ledger network to add the hash to a distributed ledger; and c) pushing the network packet to the predetermined network coordinates in response to an internally-generated prompt; and ii) remotely monitoring at least one patient compliance parameter, comprising: a) downloading the at least a portion of the device data from a HIPAA-compliant database; b) pulling the hash and the device signature applied to the hash from the distributed ledger; c) authenticating the hash by verifying the device signature applied to the hash (for example by recovering the hash from at least the device signature applied to the hash); d) verifying the accuracy of the downloaded at least a portion of the device data against the pulled hash; and e) processing the at least a portion of the device data in combination with the use instructions to obtain the at least one patient compliance parameter.

Certain embodiments may provide, for example, a method to reduce readmission rates at healthcare facilities, comprising: i) issuing mobile medical devices, with use instructions for treating medical conditions, to patients who have visited healthcare facilities, the medical devices comprising: processors, memories in communication with the processors, data sources in communication with the processors, reference to predetermined network coordinates stored in the memories, and program codes stored in the memories, the program codes executable by the processors to perform data communication operations, the data communication operations comprising: a) processing signals from the data sources to obtain device data; b) inserting dual payloads and device signatures applied to the dual payloads into network packets, first payloads of the dual payloads comprising at least portions of the device data, second payloads of the dual payloads comprising hashes of the at least portions and device signatures applied to the hashes; and c) pushing the network packets to the predetermined network coordinates in response to internally-generated prompts; and ii) reducing at least one readmission rate at the healthcare facilities for at least one of the medical conditions, comprising: a) downloading the at least portions of the device data from at least one HIPAA-compliant database; b) pulling the hashes and the device signatures applied to the hashes from at least one distributed ledger; c) authenticating the network packets by verifying the device signatures applied to the dual payloads (for example by recovering the dual payloads from the device signatures applied to the dual payloads); d) verifying the accuracies of the downloaded at least portions of the device data against the pulled hashes; and e) processing the at least portions of the device data in combination with the use instructions to determine risks of readmission of the patients.

Certain embodiments may provide, for example, a method to reduce a readmission rate at a healthcare facility, comprising: i) issuing a mobile medical device, with use instructions for treating a medical condition, to a patient who has visited a healthcare facility, the medical device comprising: a processor, a memory in communication with the processor, a data source in communication with the processor, reference to predetermined network coordinates stored in the memory, and program code stored in the memory, the program code executable by the processor to perform data communication operations, the data communication operations comprising: a) processing signals from the data source to obtain device data; b) inserting dual payloads and device signatures applied to the dual payloads into network packets, first payloads of the dual payloads comprising at least portions of the device data, second payloads of the dual payloads comprising hashes of the at least portions and device signatures applied to the hashes; and c) pushing the network packets to the predetermined network coordinates in response to internally-generated prompts; and ii) reducing a readmission rate at the healthcare facility for the medical condition, comprising: a) downloading the at least portions of the device data from a HIPAA-compliant database; b) pulling the hashes and the device signatures applied to the hashes from a distributed ledger; c) authenticating the network packets, comprising verifying the device signatures applied to the dual payloads (for example by recovering the dual payloads from the device signatures applied to the dual payloads); d) verifying the accuracies of the downloaded at least portions of the device data against the pulled hashes; and e) processing the at least portions of the device data in combination with the use instructions to determine risks of readmission of the patient.

Certain embodiments may provide, for example, a method to reduce a readmission rate at a healthcare facility, comprising: i) issuing a mobile medical device, with use instructions for treating a medical condition, to a patient who has visited a healthcare facility, the medical device comprising: a processor, a memory in communication with the processor, a data source in communication with the processor, reference to predetermined network coordinates stored in the memory, and program code stored in the memory, the program code executable by the processor to perform data communication operations, the data communication operations comprising: a) processing signals from the data source to obtain device data; b) inserting a dual payload and a device signature applied to the dual payload into a network packet, a first payload of the dual payload comprising at least a portion of the device data, a second payload of the dual payload comprising a hash of the at least a portion and a device signature applied to the hash; and c) pushing the network packet to the predetermined network coordinates in response to an internally-generated prompt; and ii) reducing a readmission rate at the healthcare facility for the medical condition, comprising: a) downloading the at least a portion of the device data from a HIPAA-compliant database; b) pulling the hash and the device signature applied to the hash from a distributed ledger; c) authenticating the hash by verifying the device signature applied to the hash (for example by recovering the hash from at least the device signature applied to the hash); d) verifying the accuracy of the downloaded at least a portion of the device data against the pulled hash; and e) processing the at least a portion of the device data in combination with the use instructions to determine a risk of readmission of the patient.

Certain embodiments may provide, for example, a method for a healthcare provider to improve remote patient compliance monitoring of use of mobile medical devices by patients, comprising: i) downloading at least portions of timing and duration-of-use data for the mobile medical devices from at least one HIPAA-compliant database; ii) pulling hashes and first digital signatures from at least one distributed ledger; iii) authenticating the hashes, comprising verifying the first digital signatures applied to the hashes using public keys assigned to the mobile medical devices (for example by recovering the hashes from the first digital signatures using the public keys assigned to the mobile medical devices); iv) verifying the accuracy of the at least portions of downloaded timing and duration-of-use data against the pulled hashes; v) processing the at least portions of downloaded timing and duration-of-use data in combination with use instructions for the mobile medical devices to detect non-compliant uses of the mobile medical devices; vi) assigning a level of risk associated with diagnosed health conditions of the patients due to the non-compliant uses; and vii) triggering an intervention protocol if the level of risk exceeds a pre-determined threshold.

Certain embodiments may provide, for example, a method for a healthcare provider to improve remote patient compliance monitoring of use of a mobile medical device by a patient, comprising: i) downloading timing and duration-of-use data for the mobile medical device from a HIPAA-compliant database; ii) pulling at least one hash and at least a first digital signature from a distributed ledger; iii) authenticating the at least one hash, comprising verifying the at least a first digital signature applied to the hash using a public key assigned to the mobile medical device (for example by recovering the hash from the at least a first digital signature using a public key assigned to the mobile medical device); iv) verifying the accuracy of the downloaded timing and duration-of-use data against the pulled at least one hash; v) processing the downloaded timing and duration-of-use data in combination with use instructions for the mobile medical device to detect non-compliant use of the mobile medical device; vi) assigning a level of risk associated with at least one diagnosed health condition of a patient due to the uninstructed use; and vii) triggering an intervention protocol if the level of risk exceeds a pre-determined threshold.

Certain embodiments may provide, for example, a method to provide patient compliance reporting to a healthcare agent. In certain embodiments, for example, the method may comprise downloading selected information from at least one HIPAA-compliant database. In certain embodiments, for example, the method may comprise pulling hashes and first digital signatures from a distributed ledger. In certain embodiments, for example, the method may comprise authenticating the hashes, comprising verifying the first digital signatures applied to the hashes using public keys assigned to the mobile medical devices (for example by recovering the hashes from the first digital signatures using public keys assigned to mobile medical devices). In certain embodiments, for example, the method may comprise verifying the accuracy of the downloaded selected information, comprising: regenerating the pulled hashes from the selected information. In certain embodiments, for example, the method may comprise preparing de-identified patient compliance summaries, comprising: processing the selected information, wherein the de-identified patient compliance summaries may be indexed by the public keys. In certain embodiments, for example, the method may comprise communicating de-identified patient compliance summaries to at least one healthcare agent.

A. In certain embodiments, for example, the method may be provided by a reporting service. In certain embodiments, for example, the reporting service may comprise a healthcare platform-as-a-service. In certain embodiments, for example, the reporting service may comprise an interface (for example an application programming interface) to a healthcare platform-as-a-service. In certain embodiments, for example, the reporting service may comprise a direct or indirect interface to at least one health insurance database. In certain embodiments, for example, the reporting service may comprise an interface to a benefits management platform. In certain embodiments, for example, the reporting service may comprise an interface to an identify management platform. In certain embodiments, for example, the reporting service may comprise an interface to a payment risk platform. In certain embodiments, for example, the reporting service may comprise an interface to a patient access platform. In certain embodiments, for example, the reporting service may comprise an interface to a claims management platform.

In certain embodiments, for example, the selected information may comprise timing and duration-of-use data for mobile medical devices.

B. In certain embodiments, for example, the preparing de-identified patient compliance summaries may comprise: processing the selected information in combination with treatment instructions for patients.

C. In certain embodiments, for example, the method may further comprise: passing at least a portion of the selected information through one or more computer models to obtain one or more data trends. In certain embodiments, for example, the method may further comprise: passing the one or more data trends to the at least one healthcare agent.

Certain embodiments may provide, for example, a method for managing patient compliance. In certain embodiments, for example, the method may comprise issuing mobile medical devices, with use instructions for treating medical conditions, to patients who have visited healthcare facilities, the medical devices comprising: processors, memories in communication with the processors, data sources in communication with the processors, reference to predetermined network coordinates stored in the memories, and program codes stored in the memories, the program codes executable by the processors to perform data communication operations, the data communication operations comprising: a) processing signals from the data sources to obtain device data; b) inserting dual payloads and device signatures applied to the dual payloads into network packets, first payloads of the dual payloads comprising at least portions of the device data, second payloads of the dual payloads comprising hashes of the at least portions and device signatures applied to the hashes; and c) pushing the network packets to the predetermined network coordinates in response to internally-generated prompts. In certain embodiments, for example, the method may comprise modifying at least one course of treatment for at least one of the medical conditions, comprising: a) receiving at least one report, the at least one report comprising de-identified summaries of the at least portions of the device data; b) processing the at least one report to identify at least one patient having at least one health risk factor that is greater than at least one predetermined threshold; and c) modifying at least one course of treatment of the at least one patient.

A. In certain embodiments, for example, the processing may comprise processing the at least one report in combination with the use instructions. In certain embodiments, for example, the at least one course of treatment may be modified to reduce at least one readmission rate for at least at one of the medical conditions at least one of the healthcare facilities.

Certain embodiments may provide, for example, a method to provide patient compliance reporting to a healthcare agent, comprising: i) downloading selected information from at least one HIPAA-compliant database; ii) pulling hashes and first digital signatures from a distributed ledger; iii) authenticating the hashes, comprising verifying the first digital signatures applied to the hashes using public keys assigned to the mobile medical devices (for example by recovering the hashes from the first digital signatures using the public keys assigned to mobile medical devices); iv) verifying the accuracy of the downloaded selected information, comprising: regenerating the pulled hashes from the selected information; v) preparing de-identified patient compliance summaries, comprising: processing the selected information, wherein the de-identified patient compliance summaries may be indexed by the public keys; and vi) communicating de-identified patient compliance summaries to at least one healthcare agent.

Certain embodiments may provide, for example, a method for managing patient compliance, comprising: i) issuing mobile medical devices, with use instructions for treating medical conditions, to patients who have visited healthcare facilities, the medical devices comprising: processors, memories in communication with the processors, data sources in communication with the processors, reference to predetermined network coordinates stored in the memories, and program codes stored in the memories, the program codes executable by the processors to perform data communication operations, the data communication operations comprising: a) processing signals from the data sources to obtain device data; b) inserting dual payloads and device signatures applied to the dual payloads into network packets, first payloads of the dual payloads comprising at least portions of the device data, second payloads of the dual payloads comprising hashes of the at least portions and device signatures applied to the hashes; and c) pushing the network packets to the predetermined network coordinates in response to internally-generated prompts; and ii) modifying at least one course of treatment for at least one of the medical conditions, comprising: a) receiving at least one report, the at least one report comprising de-identified summaries of the at least portions of the device data; b) processing the at least one report to identify at least one patient having at least one health risk factor that is greater than at least one predetermined threshold; and c) modifying at least one course of treatment of the at least one patient.

A system for remote management of patient compliance, comprising: a mobile medical device. In certain embodiments, for example, the mobile medical device may comprise a processor. In certain embodiments, for example, the mobile medical device may comprise a memory in communication with the processor. In certain embodiments, for example, the mobile medical device may comprise a data source in communication with the processor. In certain embodiments, for example, the mobile medical device may comprise a reference to predetermined network coordinates stored in the memory. In certain embodiments, for example, the mobile medical device may comprise program code stored in the memory, the program code executable by the processor to perform data communication operations, the data communication operations comprising: a) processing signals from the data source to obtain device data; b) inserting dual payloads and device signatures applied to the dual payloads into network packets, first payloads of the dual payloads comprising at least portions of the device data, second payloads of the dual payloads comprising hashes of the at least portions and device signatures applied to the hashes; and c) pushing the network packets to the predetermined network coordinates in response to internally-generated prompts.

A. In certain embodiments, for example, the mobile medical device may comprise a key signing chip having a private key embedded on the key signing chip. In certain embodiments, for example, the data communication operations may comprise causing the key signing chip to create digital signatures using the private key for at least portions of the dual payloads (for example the device signatures) and/or the hashes.

B. In certain embodiments, for example, the data source may comprise a sensor. In certain embodiments, for example, the data source may comprise a dispensing element. In certain embodiments, for example, the data source may comprise an electronic circuit. In certain embodiments, for example, the mobile medical device may further comprise an activation member (for example a depressible button) configured to trigger data gathering from the data source. In certain embodiments, for example, the mobile medical device may further comprise an actuator configured to indicate to a user that a predetermined condition has occurred. In certain embodiments, for example, the mobile medical device may further comprise a wireless network interface. In certain embodiments, for example, the mobile medical device may further comprise a power source (for example a battery such as a rechargeable battery). In certain embodiments, for example, the mobile medical device may further comprise a private key (for example a cryptographically generated private key) stored in the memory. In certain embodiments, for example, the communication operations may further comprise using the private key to generate the device signatures applied to the hashes.

C. In certain embodiments, for example, the data communication operations may comprise logging the device data in the memory. In certain embodiments, for example, the data communication operations may comprise retaining the logged device data in the memory until network packets containing the device data have been pushed. In certain embodiments, for example, the data communication operations may comprise detecting availability of a network (for example a wireless network such as a cellular network). In certain embodiments, for example, the data communication operations may comprise encrypting at least portions of the dual payloads. In certain embodiments, for example, the data communication operations may comprise monitoring how much of the device data is stored in the memory. In certain embodiments, for example, the data communications may comprise triggering the inserting and/or the pushing when the device data stored in memory and/or the dual payloads stored in memory exceed a predetermined size.

D. In certain embodiments, for example, the program code may be configured to contact a remote device to check for an update to the program code. In certain embodiments, the contacting may be strictly initiated by the mobile medical device. In certain embodiments, for example, the contacting may occur periodically (for example at predetermined intervals). In certain embodiments, for example, the program code may be configured to contact a remote device to check for an update (for example a change) to the predetermined network coordinates. In any of the foregoing embodiments, for example, the contacting and updating may comprise a pull mechanism initiated by the mobile medical device.

E. In certain embodiments, for example, the system may further comprise: a computing infrastructure, the computing infrastructure comprising: i) the predetermined network coordinates; ii) the HIPAA-compliant database configured to receive the device data; and iii) a list of public keys containing a public key for the mobile medical device, the public key for the mobile medical device effective to recover the hashes from the device signatures applied to the hashes; and iv) at least one access point (for example at least one wallet, at least one peer, at least one executable smart contract) for communication with a distributed ledger network, the at least one access point configured to push instructions to a distributed ledger to add the hashes to the distributed ledger without any patient identification information. In certain embodiments, for example, the system may maintain a list of authorized nodes (for example the mobile medical device) and associated device identification codes (or public keys). In certain embodiments, for example, the system may be configured to detect and to prevent communications with (or ignore network communications from) nodes that are not authorized notes. In certain embodiments, for example, the system may enforce a payment scheme whereby processing one or more of the dual payloads requires payment (for example payment in cryptocurrency).

F. In certain embodiments, for example, the computing infrastructure may further comprise: i) a further processor; ii) a private key; and iii) instructions executable by the further processor to use the private key to generate digital signatures applied to the device signatures applied to the hashes.

G. In certain embodiments, for example, the mobile medical device may be a handheld mobile medical device for use comprising use outside a healthcare facility setting. In certain embodiments, the mobile medical device may be for use comprising (for example consisting) use inside a healthcare facility (for example a mobile medical device configured to provide vital sign data to a hospital monitoring system). In certain embodiments, the mobile medical device may be for use comprising: use inside a home (for example for use by persons confined to a home).

A system for remote management of patient compliance, comprising: a mobile medical device, comprising: i) a processor; ii) a memory in communication with the processor; iii) a data source in communication with the processor; iv) a reference to predetermined network coordinates stored in the memory; and v) program code stored in the memory, the program code executable by the processor to perform data communication operations, the data communication operations comprising: a) processing signals from the data source to obtain device data; b) inserting dual payloads and device signatures applied to the dual payloads into network packets, first payloads of the dual payloads comprising at least portions of the device data, second payloads of the dual payloads comprising hashes of the at least portions and device signatures applied to the hashes; and c) pushing the network packets to the predetermined network coordinates in response to internally-generated prompts.

Certain embodiments may provide, for example, a method to monitor compliance with prescribed usage of a device. In certain embodiments, for example, the method may comprise assigning a device identification code and prescribed usage parameters for the device to an intended user of the device. In certain embodiments, for example, the method may comprise storing data (or a hash of the data) for the device in a distributed ledger, the data for the device comprising the device identification code and a log of device usage data received from the device, the data for the device exclusive of identifying information about the intended user. In certain embodiments, for example, the method may comprise retrieving the log of device usage data from the distributed ledger. In certain embodiments, for example, the method may comprise comparing at least a portion of the retrieved log of device usage with the prescribed usage parameters. In certain embodiments, for example, the method may comprise sending results of the comparison, with an identifier for the intended user, to an interpreter of the results.

A. In certain embodiments, for example, the log of device usage data may comprise at least one of time, duration, and frequency of usage of the device. In certain embodiments, for example, the log of device usage data may be transmitted from the device or a cellular radio in communication with the device via a cellular network. In certain embodiments, for example, the received data may be at least one of a voice message, text message, pager notification, electronic mail, Internet message, private network communication, and protocol communication. In certain embodiments, for example, the log of device usage data may be retrieved via the public internet. In certain embodiments, for example, the comparing may be periodic. In certain embodiments, for example, the comparing may be performed within 2 days (or within 1 day, within 5 days, within 10 days, or within 30 days) of the storing. In certain embodiments, for example, the method may further comprise: retrieving the prescribed usage parameters from an electronic health records (EHR) database. In certain embodiments, for example, the sending may be triggered if the log of device usage indicates non-compliant usage or lack of usage of the device by the intended user. In certain embodiments, for example, the interpreter may be a healthcare provider (for example a nurse, a doctor, and/or a clinician). In certain embodiments, for example, the interpreter may be assigned when the device identification code is assigned.

Certain embodiments may provide, for example, a method to manage operation of a mobile device. In certain embodiments, for example, the method may comprise transmitting operating instructions for a mobile device to an intended operator of the device, and recording the operating instructions in a first database. In certain embodiments, for example, the method may comprise receiving time series of operating data from the device when deployed via a pre-configured secure connection, and publishing the received time series to a second database via the public Internet without identifying the intended operator, the second database comprising a distributed ledger. In certain embodiments, for example, the method may comprise periodically auditing the operating history of the device, comprising: a) retrieving the operating instructions from the first database, and retrieving at least a portion of the operating data via the public Internet from the second database; and b) comparing the retrieved at least a portion of the time series to the operating instructions. In certain embodiments, for example, the method may comprise providing updated operating instructions to the intended operator in at least one response to the periodic auditing.

Certain embodiments may provide, for example, a method to increase patient compliance with use instructions for a device prescribed to the patient. In certain embodiments, for example, the method may comprise assigning a physician, a device identification code, and use instructions for the device to a patient who has been admitted to a healthcare facility. In certain embodiments, for example, the method may comprise storing data for the device in a distributed ledger, the data for the device comprising the device identification code and a log of device usage data received from the device, the data for the device exclusive of identifying information about the intended user. In certain embodiments, for example, the method may comprise retrieving the log of device usage data (or hashes of device usage data) from the distributed ledger. In certain embodiments, for example, the method may comprise determining risk of readmission of the patient, comprising: accounting for at least a portion of the log of device usage data. In certain embodiments, for example, the method may comprise triggering an intervention protocol if the determined risk of readmission exceeds a pre-determined threshold.

A. In certain embodiments, for example, the physician may conduct a face-to-face meeting with the patient prior to the interpreting. In certain embodiments, for example, the use instructions may be present in (or derivable from) at least one of a prescription for the device and a drug label for a drug to be administered via the device. In certain embodiments, for example, at least one of the assigning and the storing may occur before or during discharge of the patient from the healthcare facility.

B. In certain embodiments, for example, the determining may be performed by the assigned physician (or a party designated by the physician such as a clinician). In certain embodiments, for example, the determining may require at least 30 minutes. In certain embodiments, for example, the determining may not involve a direct interaction between the physician and the patient (for example the determining may be remote). In certain embodiments, for example, the determining may comprise comparing the retrieved actual usage parameters with the use instructions. In certain embodiments, for example, the use instructions may be obtained from a HIPPA-compliant EHR database.

C. In certain embodiments, for example, the determining may be limited to determining the risk of readmission within 30 days of the admission.

D. In certain embodiments, for example, the determining may further comprise: accounting for at least one of disease information, prior admittance information, prior admittance facility type information, and patient demographic information for the patient.

E. In certain embodiments, for example, the log of device usage may be used to estimate an amount of medicament delivered to the patient.

Certain embodiments may provide, for example, a method to reduce operator risk. In certain embodiments, for example, the method may comprise receiving time series of operating data from a deployed mobile device, and transmitting the received time series to a distributed ledger via the public Internet, the receiving and the transmitting performed without identifying an intended operator of the device. In certain embodiments, for example, the method may comprise periodically testing for at least one risk to the intended operator, comprising: a) retrieving a portion of the time series corresponding to a prior window of operation of the device (for example a window of operation in the range of 15-40 days) from the distributed ledger; b) verifying that the portion of the time series is immutably encoded in the distributed ledger; and c) detecting incongruent operation of the device during the prior window of operation, comprising: comparing the portion of the time series with operating instructions, the operating instructions in effect during the prior window of operation. In certain embodiments, for example, the method may comprise triggering an intervention protocol if the at least one risk exceeds a predetermined threshold.

Certain embodiments may provide, for example, a method to monitor compliance with prescribed usage of a device, comprising: i) assigning a device identification code and prescribed usage parameters for the device to an intended user of the device; ii) storing data for the device in a distributed ledger, the data for the device comprising the device identification code and a log of device usage data received from the device, the data for the device exclusive of identifying information about the intended user; iii) retrieving the log of device usage data from the distributed ledger; iv) comparing at least a portion of the retrieved log of device usage with the prescribed usage parameters; and v) sending results of the comparison, with an identifier for the intended user, to an interpreter of the results.

Certain embodiments may provide, for example, a method to manage operation of a mobile device, comprising: i) transmitting operating instructions for a mobile device to an intended operator of the device, and recording the operating instructions in a first database; ii) receiving time series of operating data from the device when deployed via a pre-configured secure connection, and publishing the received time series to a second database via the public Internet without identifying the intended operator, the second database comprising a distributed ledger; iii) periodically auditing the operating history of the device, comprising: a) retrieving the operating instructions from the first database, and retrieving at least a portion of the operating data via the public Internet from the second database; and b) comparing the retrieved at least a portion of the time series to the operating instructions; and iv) providing updated operating instructions to the intended operator in at least one response to the periodic auditing.

Certain embodiments may provide, for example, a method to increase patient compliance with use instructions for a device prescribed to the patient, comprising: i) assigning a physician, a device identification code, and use instructions for the device to a patient who has been admitted to a healthcare facility; ii) storing data for the device in a distributed ledger, the data for the device comprising the device identification code and a log of device usage data received from the device, the data for the device exclusive of identifying information about the intended user; iii) retrieving the log of device usage data from the distributed ledger; iv) determining risk of readmission of the patient, comprising: accounting for at least a portion of the log of device usage data; and v) triggering an intervention protocol if the determined risk of readmission exceeds a pre-determined threshold.

Certain embodiments may provide, for example, a method for obtaining authenticated data from a mobile medical device. In certain embodiments, for example, the method may comprise registering the device to a patient, comprising: receiving patient identifying information in combination with a device registration code via the public internet. In certain embodiments, for example, the method may comprise receiving data pushed from the registered device, the data comprising: usage information and digitally-signed instructions for a distributed ledger system to record a hash of the usage information. In certain embodiments, for example, the method may comprise inserting the usage information into at least one patient record in a private database. In certain embodiments, for example, the method may comprise passing the instructions, exclusive of any patient reference, to the distributed ledger system.

A. In certain embodiments, for example, the registration code may be a single-use device registration code. In certain embodiments, for example, the method may further comprise verifying the patient has executed a HIPAA release form prior to the inserting and the passing.

Certain embodiments may provide, for example, a method for obtaining authenticated data from a mobile medical device, comprising: i) registering the device to a patient, comprising: receiving patient identifying information in combination with a device registration code via the public internet; ii) receiving data pushed from the registered device, the data comprising: usage information and digitally-signed instructions for a distributed ledger system to record a hash of the usage information; iii) inserting the usage information into at least one patient record in a private database; and iv) passing the instructions, exclusive of any patient reference, to the distributed ledger system.

Certain embodiments may provide, for example, a method for HIPAA-compliant redistribution of usage information received from a mobile medical device. In certain embodiments, for example, the method may comprise approving a party for access to the data, comprising: a) receiving an access request comprising an access code via the Internet; b) obtaining electronic contact information for a patient in a private database based on the access code; and c) passing the access request (and optionally a HIPAA release form) to the patient followed by receiving approval from the patient. In certain embodiments, for example, the method may comprise transmitting the usage information, exclusive of any patient reference, in response to a pull request from the approved party, the pull request comprising a device identification code for the device. In certain embodiments, for example, the method may comprise pushing data from the registered device, the data comprising: usage information and digitally-signed instructions for a distributed ledger system to record a hash of the usage information. In certain embodiments, for example, the method may comprise inserting the usage information into at least one patient record in a private database. In certain embodiments, for example, the method may comprise passing the instructions, exclusive of any patient reference, to the distributed ledger system.

A. In certain embodiments, for example, the approval may be revoked by the patient. In certain embodiments, for example, the access code may be provided on a digitally scannable portion of the mobile medical device.

Certain embodiments may provide, for example, a method for HIPAA-compliant redistribution of usage information received from a mobile medical device, comprising: i) approving a party for access to the data, comprising: a) receiving an access request comprising an access code via the Internet; b) obtaining electronic contact information for a patient in a private database based on the access code; and c) passing the access request to the patient followed by receiving approval from the patient; ii) transmitting the usage information, exclusive of any patient reference, in response to a pull request from the approved party, the pull request comprising a device identification code for the device; iii) pushing data from the registered device, the data comprising: usage information and digitally-signed instructions for a distributed ledger system to record a hash of the usage information; iv) inserting the usage information into at least one patient record in a private database; and v) passing the instructions, exclusive of any patient reference, to the distributed ledger system.

Certain embodiments may provide, for example, a method for storing data from mobile medical devices. In certain embodiments, for example, the method may comprise receiving a network packet from one of the mobile medical devices, the network packet comprising a dual payload, the dual payload comprising: a) a first payload comprising a device identification code and de-identified data; and b) a second payload comprising a transaction object, the transaction object comprising: ciphertext, and a digital signature applied to the ciphertext. In certain embodiments, for example, the method may comprise matching the ciphertext to a hash of at least a portion of the de-identified data. In certain embodiments, for example, the method may comprise identifying a patient associated with the device identification code. In certain embodiments, for example, the method may comprise inserting the de-identified data into a record for the patient in a HIPAA-compliant database. In certain embodiments, for example, the method may comprise pushing the transaction object across a network to at least one peer in a distributed ledger without any patient identification information.

A. In certain embodiments, for example, the second payload may further comprise a copy of the device identification code. In certain embodiments, for example, the transaction object may comprise a copy of the device identification code. In certain embodiments, for example, a predetermined signature verifying algorithm may be applied to the dual payload, the first digital signature, and a public key corresponding to, comprising, or derived from the device identification code to authenticate the network packet. In certain embodiments, for example, the second payload may comprise a smart contract or reference to a smart contract. In certain embodiments, for example, the smart contract may comprise a write function. In certain embodiments, for example, the smart contract functionality may be limited to write functionality. In certain embodiments, for example, the transaction object may comprise a smart contract or a reference to a smart contract. In certain embodiments, for example, the second payload may be exclusive of patient identification information.

B. In certain embodiments, for example, the digital signature may be generated by a key signing chip onboard the mobile medical device. In certain embodiments, for example, the key signing chip may comprise a private key, the private key used to generate the digital signature. In certain embodiments, for example, the device identification code may be a public key paired to a private key, the private key used to generate the digital signature. In certain embodiments, for example, the method may further comprise: verifying the digital signature, comprising: passing a series of parameters through one or more predetermined digital signature verification functions, the series of parameters comprising: the ciphertext and a parameter derived from the device identification code. In certain embodiments, for example, the parameter derived from the device identification code may be at least a portion of the device identification code. In certain embodiments, for example, the device identification code may correspond to, comprise, or be used to derive a public key, the public key corresponding to a private key used to form the digital signature. In certain embodiments, for example, the de-identified data may comprise at least one timestamp and at least one duration-of-use for the mobile medical device.

C. In certain embodiments, for example, the method may be performed by one or more software running on one or more nodes in a peer-to-peer network. In certain embodiments, for example, the peer-to-peer network may comprise a distributed database. In certain embodiments, for example, the peer-to-peer network may conform to an InterPlanetary File System (IPFS) protocol. In certain embodiments, for example, the method may utilize one or more web services. In certain embodiments, for example, the transaction object may be processable to add the ciphertext to a distributed ledger. In certain embodiments, for example, the transaction object may further comprise a smart contract or a reference to a smart contract. In certain embodiments, for example, the transaction object may further comprise a quantity of a cryptocurrency. In certain embodiments, for example, the transaction object may further comprise instructions to transfer at least a portion of the quantity of a cryptocurrency to pay for network access for the mobile medical device. In certain embodiments, for example, the HIPAA-compliant database may be a distributed database. In certain embodiments, for example, the HIPAA-compliant database may comprise a distributed hash table (DHT). In certain embodiments, for example, the HIPAA-compliant database may comprise a distributed hash table (DHT). In certain embodiments, for example, the HIPAA-compliant database may comprise a distributed database configured according to Inter-Planetary File System (IPFS) protocol. In certain embodiments, for example, the HIPAA-compliant database may comprise a b-tree data structure. In certain embodiments, for example, the transaction object may be pushed to the distributed ledger from a wallet. In certain embodiments, for example, the de-identified data may be pushed to the distributed ledger from a peer in a distributed ledger network. In certain embodiments, for example, the pushing may comprise invoking a smart contract. In certain embodiments, for example, the pushing may comprise providing a quantity of a cryptocurrency. In certain embodiments, for example, the network packet may be independently pushed from the one of the mobile medical devices. In certain embodiments, for example, the pushing may comprise passing the transaction object to a peer in the distributed ledger network. In certain embodiments, for example, the transaction object may further comprise a digital signature for the HIPAA-compliant database, the digital signature applied at least to the ciphertext.

D. In certain embodiments, for example, the method may further comprise authorizing the one of the mobile medical devices to transmit the network packet, the authorizing comprising registering the one of the mobile medical devices via an electronic interface. In certain embodiments, for example, the registering the one of the mobile medical devices may comprise receiving one or more executed HIPAA release forms from a patient. In certain embodiments, for example, the registering the one of the mobile medical devices may comprise associating the device identification code with patient identification information in the HIPAA-compliant database.

E. In certain embodiments, for example, the method may further comprise authorizing access to at least one of a HIPAA-compliant database record for a patient, a block in a blockchain referencing the ciphertext in the distributed ledger, and the ciphertext in the distributed ledger. In certain embodiments, for example, the authorizing access may comprise a) forwarding an access request from a third party to the patient; and b) receiving access permission for the third party from the patient. In certain embodiments, for example, the identity of a third party receiving access authorization may be stored in a specified distributed ledger. In certain embodiments, for example, the specified distributed ledger may be checked when the third party attempts to access the at least one of the HIPAA-compliant database record, the block in a blockchain referencing the hash in the distributed ledger, and the hash in the distributed ledger.

Certain embodiments may provide, for example, a method for processing data from mobile medical devices. In certain embodiments, for example, the method may comprise receiving a network packet from one of the mobile medical devices, the network packet comprising a dual payload, a first payload of the dual payload comprising de-identified data, a second payload of the dual payload comprising a digital signature and a hash. In certain embodiments, for example, the method may comprise authenticating the network packet by verifying the digital signature using a member of a list of public keys assigned to authorized data sources. In certain embodiments, for example, the method may comprise using the member of the list of public keys to identify a patient. In certain embodiments, for example, the method may comprise inserting the anonymous data into a record for the patient in a HIPAA-compliant database. In certain embodiments, for example, the method may comprise pushing the anonymous data to a distributed ledger without any patient identification information.

A. In certain embodiments, for example, the hash may be formed by passing at least a portion of the de-identified data through a predetermined hash function. In certain embodiments, for example, the hash may be formed on the one of the mobile medical devices.

Certain embodiments may provide, for example, a method for processing data from mobile medical devices. In certain embodiments, for example, the method may comprise receiving network packets, the network packets comprising dual payloads, first payloads of the dual payloads comprising de-identified data, second payloads of the dual payloads comprising digital signatures and hashes. In certain embodiments, for example, the method may comprise authenticating the network packets by verifying the digital signatures using members of a list of public keys assigned to authorized data sources. In certain embodiments, for example, the method may comprise using the members of the list of public keys to identify patients. In certain embodiments, for example, the method may comprise inserting the anonymous data into records for the patients in at least one HIPAA-compliant database. In certain embodiments, for example, the method may comprise pushing the anonymous data to at least one distributed ledger without any patient identification information.

Certain embodiments may provide, for example, a method for storing data from mobile medical devices, comprising: i) receiving a network packet from one of the mobile medical devices, the network packet comprising a dual payload, the dual payload comprising: a) a first payload comprising a device identification code and de-identified data; and b) a second payload comprising a transaction object, the transaction object comprising: ciphertext, and a digital signature applied to the ciphertext; ii) matching the ciphertext to a hash of at least a portion of the de-identified data; iii) identifying a patient associated with the device identification code; iv) inserting the de-identified data into a record for the patient in a HIPAA-compliant database; and v) pushing the transaction object across a network to at least one peer in a distributed ledger without any patient identification information.

Certain embodiments may provide, for example, a method for processing data from mobile medical devices, comprising: i) receiving a network packet from one of the mobile medical devices, the network packet comprising a dual payload, a first payload of the dual payload comprising de-identified data, a second payload of the dual payload comprising a digital signature and a hash; ii) authenticating the network packet by verifying the digital signature using a member of a list of public keys assigned to authorized data sources; iii) using the member of the list of public keys to identify a patient; iv) inserting the anonymous data into a record for the patient in a HIPAA-compliant database; and v) pushing the anonymous data to a distributed ledger without any patient identification information.

Certain embodiments may provide, for example, a method for processing data from mobile medical devices, comprising: i) receiving network packets, the network packets comprising dual payloads, first payloads of the dual payloads comprising de-identified data, second payloads of the dual payloads comprising digital signatures and hashes; ii) authenticating the network packets by verifying the digital signatures using members of a list of public keys assigned to authorized data sources; iii) using the members of the list of public keys to identify patients; iv) inserting the anonymous data into records for the patients in at least one HIPAA-compliant database; and v) pushing the anonymous data to at least one distributed ledger without any patient identification information

Certain embodiments may provide, for example, a method for remote third-party monitoring of data from a mobile medical device. In certain embodiments, for example, the method may comprise issuing a mobile medical device, with use instructions for treating a medical condition, to a subject, the medical device comprising: a processor, a memory in communication with the processor, a data source in communication with the processor, reference to predetermined network coordinates stored in the memory, and program code stored in the memory, the program code executable by the processor to perform data communication operations, the data communication operations comprising: a) processing signals from the data source to obtain device data; b) inserting dual payloads into network packets, first payloads of the dual payloads comprising at least portions of the device data, second payloads of the dual payloads comprising hashes of the at least portions of the device data and device signatures, the device signatures obtained by applying predetermined digital signing algorithms to the hashes; and c) pushing the network packets to the predetermined network coordinates in response to internally-generated prompts. In certain embodiments, for example, the method may comprise providing access to a third party to remotely monitor the device data, the remote monitoring comprising: a) downloading at least portions of the device data from a HIPAA-compliant database; b) pulling the hashes and the device signatures applied to the hashes from a distributed ledger; c) verifying the device signatures applied to the hashes; and d) checking the accuracies of the downloaded at least portions of the device data using the pulled hashes.

A. In certain embodiments, for example, the issuing may further comprise: providing a release to the subject to provide the third party with access to the device data. In certain embodiments, for example, the providing access may be proximate the time of the issuing. In certain embodiments, for example, the providing access may occur at a point of sale of the mobile medical device. In certain embodiments, for example, the providing access may comprise scanning a bar code on the mobile medical device. In certain embodiments, for example, the providing access may be independent of any activation of the mobile medical device by the subject. In certain embodiments, for example, the providing access may be prior to any activation of the mobile medical device.

B. In certain embodiments, for example, the third party may be a manufacturer of the mobile medical device. In certain embodiments, for example, the third party may provide a warranty for mobile medical device. In certain embodiments, for example, the third party may be an insurance company. In certain embodiments, for example, the third party may perform data analysis on the device data. In certain embodiments, for example, the third party may be a member of a predetermined list of third parties with permission to access the device data.

C. In certain embodiments, for example, the predetermined list may be stored in a further distributed ledger. In certain embodiments, for example, the further distributed ledger may be checked when the third party attempts to access the at least one of the HIPAA-compliant database record, a block in a blockchain referencing the hash in the distributed ledger, and the hash in the distributed ledger. In certain embodiments, for example, the attempt to access may be digitally signed by the third party. In certain embodiments, for example, the further distributed ledger may be the distributed ledger.

Certain embodiments may provide, for example, a method for remote management of patient compliance. In certain embodiments, for example, the method may comprise issuing a mobile medical device, with use instructions for treating a medical condition, to a patient, the medical device comprising: a processor, a memory in communication with the processor, a data source in communication with the processor, reference to predetermined network coordinates stored in the memory, and program code stored in the memory, the program code executable by the processor to perform data communication operations, the data communication operations comprising: a) processing signals from the data source to obtain device data; b) inserting dual payloads into network packets, first payloads of the dual payloads comprising at least portions of the device data, second payloads of the dual payloads comprising hashes of the at least portions of the device data and device signatures, the device signatures obtained by applying predetermined digital signing algorithms to the hashes; and c) pushing the network packets to the predetermined network coordinates in response to internally-generated prompts. In certain embodiments, for example, the method may comprise remotely monitoring at least one patient compliance parameter, comprising: a) downloading the at least portions of the device data from a HIPAA-compliant database; b) pulling the hashes and the device signatures applied to the hashes from a distributed ledger; c) verifying the device signatures applied to the hashes; d) checking the accuracies of the downloaded at least portions of the device data using the pulled hashes; and e) processing the at least portions of the device data in combination with the use instructions to obtain the at least one patient compliance parameter.

A. In certain embodiments, for example, the device signatures may be generated onboard the mobile medical device using a private key stored on the mobile medical device. In certain embodiments, for example, the first payloads of the dual payloads may further comprise a device identification code for the mobile medical device. In certain embodiments, for example, at least one of the device signatures may be verified by passing the device identification code, or a parameter derived from the device identification code, the at least one of the digital signatures, and one of the hashes through one or more digital signature verification functions.

B. In certain embodiments, for example, the mobile medical device may further comprise a key signing chip configured to apply the predetermined digital signing algorithms using a private key stored on the mobile medical device as an input. In certain embodiments, for example, the private key may be embedded in the key signing chip.

C. In certain embodiments, for example, the mobile medical device may dispense an opioid drug. In certain embodiments, for example, the mobile medical device may dispense a chemotherapeutic agent. In certain embodiments, for example, the method may further comprise: a) detecting overuse of the mobile medical device based on the at least one patient compliance parameter, and b) transmitting a signal to the mobile medical device to prevent further use of the device. In certain embodiments, for example, the method may further comprise: a) detecting a predetermined number of uses of the mobile medical device has occurred, the predetermined number based on a medical determination; and b) transmitting a signal to the mobile medical device to prevent further use of the mobile medical device until a further signal may be received that authorizes further use of the mobile medical device.

Certain embodiments may provide, for example, a method for remote management of patient compliance. In certain embodiments, for example, the method may comprise issuing mobile medical devices, with use instructions for treating medical conditions, to patients, the medical devices comprising: processors, memories in communication with the processors, data sources in communication with the processors, reference to predetermined network coordinates stored in the memories, private digital signing keys, device identification codes assigned to the private digital signing keys, and program codes stored in the memories, the program codes executable by the processors to perform data communication operations, the data communication operations comprising: a) processing signals from the data sources to obtain device data; b) inserting dual payloads into network packets, first payloads of the dual payloads comprising the device identification codes and at least portions of the device data, second payloads of the dual payloads comprising hashes of the at least portions of the device data and device signatures applied to the hashes, the device signatures obtained by applying digital signing algorithms to the hashes; and c) pushing the network packets to the predetermined network coordinates in response to internally-generated prompts. In certain embodiments, for example, the method may comprise remotely monitoring at least one patient compliance parameter, comprising: a) downloading the at least portions of the device data from at least one HIPAA-compliant database; b) pulling the hashes and the device signatures applied to the hashes from at least one distributed ledger; c) verifying the device signatures applied to the hashes; d) checking the accuracies of the downloaded at least portions of the device data using the pulled hashes; and e) processing the at least portions of the device data in combination with the use instructions to obtain the at least one patient compliance parameter.

Certain embodiments may provide, for example, a method to reduce readmission rates at healthcare facilities. In certain embodiments, for example, the method may comprise issuing mobile medical devices, with use instructions for treating medical conditions, to patients who have visited the healthcare facilities, the medical devices comprising: processors, memories in communication with the processors, data sources in communication with the processors, reference to predetermined network coordinates stored in the memories, private digital signing keys, device identification codes assigned to the private digital signing keys, and program codes stored in the memories, the program codes executable by the processors to perform data communication operations, the data communication operations comprising: a) processing signals from the data sources to obtain device data; b) inserting dual payloads into network packets, first payloads of the dual payloads comprising the device identification codes for the mobile medical devices and at least portions of the device data, second payloads of the dual payloads comprising hashes of the at least portions of the device data and device signatures applied to the hashes, the device signatures obtained by applying digital signing algorithms to the hashes on the mobile medical devices; and c) pushing the network packets to the predetermined network coordinates in response to internally-generated prompts. In certain embodiments, for example, the method may comprise reducing at least one readmission rate at the healthcare facilities for at least one of the medical conditions, comprising: a) downloading the at least portions of the device data from at least one HIPAA-compliant database; b) pulling the hashes and the device signatures applied to the hashes from at least one distributed ledger; c) verifying the device signatures applied to the hashes; d) checking the accuracies of the downloaded at least portions of the device data using the pulled hashes; and e) processing the at least portions of the device data in combination with the use instructions to determine risks of readmission of the patients.

Certain embodiments may provide, for example, a method for a healthcare provider to improve remote patient compliance monitoring of use of mobile medical devices by patients. In certain embodiments, for example, the method may comprise downloading at least portions of timing and duration-of-use data for the mobile medical devices from at least one HIPAA-compliant database. In certain embodiments, for example, the method may comprise pulling hashes and digital signatures applied to the hashes from at least one distributed ledger. In certain embodiments, for example, the method may comprise verifying the digital signatures, comprising: passing the hashes, the digital signatures, and public keys assigned to the mobile medical devices through one or more digital signature verifying functions. In certain embodiments, for example, the method may comprise checking the accuracy of the at least portions of downloaded timing and duration-of-use data using the pulled hashes. In certain embodiments, for example, the method may comprise processing the at least portions of downloaded timing and duration-of-use data in combination with use instructions for the mobile medical devices to detect non-compliant uses of the mobile medical devices. In certain embodiments, for example, the method may comprise assigning levels of risks associated with diagnosed health conditions of the patients due to the non-compliant uses. In certain embodiments, for example, the method may comprise triggering intervention protocol if the levels of risk exceed pre-determined thresholds.

A. In certain embodiments, for example, the non-compliant uses may comprise overuses of the mobile medical devices. In certain embodiments, for example, the non-compliant uses may comprise underuses of the mobile medical devices. In certain embodiments, for example, the levels of risk may comprise risks of deterioration of one or more of the diagnosed heath conditions.

Certain embodiments may provide, for example, a method for a healthcare provider to improve remote patient compliance monitoring of use of a mobile medical device by a patient. In certain embodiments, for example, the method may comprise downloading timing and duration-of-use data for the mobile medical device from a HIPAA-compliant database. In certain embodiments, for example, the method may comprise pulling at least one hash and at least one digital signature applied to the at least one hash from a distributed ledger. In certain embodiments, for example, the method may comprise verifying the at least one hash, comprising: passing the at least one hash, the at least one digital signatures, and a public key assigned to the mobile medical device through one or more digital signature verifying functions. In certain embodiments, for example, the method may comprise checking the accuracy of the downloaded timing and duration-of-use data using the pulled at least one hash. In certain embodiments, for example, the method may comprise processing the downloaded timing and duration-of-use data in combination with use instructions for the mobile medical device to detect non-compliant use of the mobile medical device. In certain embodiments, for example, the method may comprise assigning a level of risk associated with at least one diagnosed health condition of a patient due to the uninstructed use. In certain embodiments, for example, the method may comprise triggering an intervention protocol if the level of risk exceeds a pre-determined threshold.

A. In certain embodiments, for example, the non-compliant use may comprise overuse of the mobile medical device. In certain embodiments, for example, the non-compliant use may comprise underuse of the mobile medical device. In certain embodiments, for example, the level of risk may be a risk of deterioration of the at least one diagnosed heath condition.

Certain embodiments may provide, for example, a method for remote third-party monitoring of data from a mobile medical device, comprising: i) issuing a mobile medical device, with use instructions for treating a medical condition, to a subject, the medical device comprising: a processor, a memory in communication with the processor, a data source in communication with the processor, reference to predetermined network coordinates stored in the memory, and program code stored in the memory, the program code executable by the processor to perform data communication operations, the data communication operations comprising: a) processing signals from the data source to obtain device data; b) inserting dual payloads into network packets, first payloads of the dual payloads comprising at least portions of the device data, second payloads of the dual payloads comprising hashes of the at least portions of the device data and device signatures, the device signatures obtained by applying predetermined digital signing algorithms to the hashes; and c) pushing the network packets to the predetermined network coordinates in response to internally-generated prompts; and ii) providing access to a third party to remotely monitor the device data, the remote monitoring comprising: a) downloading at least portions of the device data from a HIPAA-compliant database; b) pulling the hashes and the device signatures applied to the hashes from a distributed ledger; c) verifying the device signatures applied to the hashes; and d) checking the accuracies of the downloaded at least portions of the device data using the pulled hashes.

Certain embodiments may provide, for example, a method for remote management of patient compliance, comprising: i) issuing a mobile medical device, with use instructions for treating a medical condition, to a patient, the medical device comprising: a processor, a memory in communication with the processor, a data source in communication with the processor, reference to predetermined network coordinates stored in the memory, and program code stored in the memory, the program code executable by the processor to perform data communication operations, the data communication operations comprising: a) processing signals from the data source to obtain device data; b) inserting dual payloads into network packets, first payloads of the dual payloads comprising at least portions of the device data, second payloads of the dual payloads comprising hashes of the at least portions of the device data and device signatures, the device signatures obtained by applying predetermined digital signing algorithms to the hashes; and c) pushing the network packets to the predetermined network coordinates in response to internally-generated prompts; and ii) remotely monitoring at least one patient compliance parameter, comprising: a) downloading the at least portions of the device data from a HIPAA-compliant database; b) pulling the hashes and the device signatures applied to the hashes from a distributed ledger; c) verifying the device signatures applied to the hashes; d) checking the accuracies of the downloaded at least portions of the device data using the pulled hashes; and e) processing the at least portions of the device data in combination with the use instructions to obtain the at least one patient compliance parameter.

Certain embodiments may provide, for example, a method for remote management of patient compliance, comprising: i) issuing mobile medical devices, with use instructions for treating medical conditions, to patients, the medical devices comprising: processors, memories in communication with the processors, data sources in communication with the processors, reference to predetermined network coordinates stored in the memories, private digital signing keys, device identification codes assigned to the private digital signing keys, and program codes stored in the memories, the program codes executable by the processors to perform data communication operations, the data communication operations comprising: a) processing signals from the data sources to obtain device data; b) inserting dual payloads into network packets, first payloads of the dual payloads comprising the device identification codes and at least portions of the device data, second payloads of the dual payloads comprising hashes of the at least portions of the device data and device signatures applied to the hashes, the device signatures obtained by applying digital signing algorithms to the hashes; and c) pushing the network packets to the predetermined network coordinates in response to internally-generated prompts; and ii) remotely monitoring at least one patient compliance parameter, comprising: a) downloading the at least portions of the device data from at least one HIPAA-compliant database; b) pulling the hashes and the device signatures applied to the hashes from at least one distributed ledger; c) verifying the device signatures applied to the hashes; d) checking the accuracies of the downloaded at least portions of the device data using the pulled hashes; and e) processing the at least portions of the device data in combination with the use instructions to obtain the at least one patient compliance parameter.

Certain embodiments may provide, for example, a method to reduce readmission rates at healthcare facilities, comprising: i) issuing mobile medical devices, with use instructions for treating medical conditions, to patients who have visited the healthcare facilities, the medical devices comprising: processors, memories in communication with the processors, data sources in communication with the processors, reference to predetermined network coordinates stored in the memories, private digital signing keys, device identification codes assigned to the private digital signing keys, and program codes stored in the memories, the program codes executable by the processors to perform data communication operations, the data communication operations comprising: a) processing signals from the data sources to obtain device data; b) inserting dual payloads into network packets, first payloads of the dual payloads comprising the device identification codes for the mobile medical devices and at least portions of the device data, second payloads of the dual payloads comprising hashes of the at least portions of the device data and device signatures applied to the hashes, the device signatures obtained by applying digital signing algorithms to the hashes on the mobile medical devices; and c) pushing the network packets to the predetermined network coordinates in response to internally-generated prompts; and ii) reducing at least one readmission rate at the healthcare facilities for at least one of the medical conditions, comprising: a) downloading the at least portions of the device data from at least one HIPAA-compliant database; b) pulling the hashes and the device signatures applied to the hashes from at least one distributed ledger; c) verifying the device signatures applied to the hashes; d) checking the accuracies of the downloaded at least portions of the device data using the pulled hashes; and e) processing the at least portions of the device data in combination with the use instructions to determine risks of readmission of the patients.

Certain embodiments may provide, for example, a method for a healthcare provider to improve remote patient compliance monitoring of use of mobile medical devices by patients, comprising: i) downloading at least portions of timing and duration-of-use data for the mobile medical devices from at least one HIPAA-compliant database; ii) pulling hashes and digital signatures applied to the hashes from at least one distributed ledger; iii) verifying the digital signatures, comprising: passing the hashes, the digital signatures, and public keys assigned to the mobile medical devices through one or more digital signature verifying functions; iv) checking the accuracy of the at least portions of downloaded timing and duration-of-use data using the pulled hashes; v) processing the at least portions of downloaded timing and duration-of-use data in combination with use instructions for the mobile medical devices to detect non-compliant uses of the mobile medical devices; vi) assigning levels of risks associated with diagnosed health conditions of the patients due to the non-compliant uses; and vii) triggering intervention protocol if the levels of risk exceed pre-determined thresholds.

Certain embodiments may provide, for example, a method for a healthcare provider to improve remote patient compliance monitoring of use of a mobile medical device by a patient, comprising: i) downloading timing and duration-of-use data for the mobile medical device from a HIPAA-compliant database; ii) pulling at least one hash and at least one digital signature applied to the at least one hash from a distributed ledger; iii) verifying the at least one hash, comprising: passing the at least one hash, the at least one digital signatures, and a public key assigned to the mobile medical device through one or more digital signature verifying functions; iv) checking the accuracy of the downloaded timing and duration-of-use data using the pulled at least one hash; v) processing the downloaded timing and duration-of-use data in combination with use instructions for the mobile medical device to detect non-compliant use of the mobile medical device; vi) assigning a level of risk associated with at least one diagnosed health condition of a patient due to the uninstructed use; and vii) triggering an intervention protocol if the level of risk exceeds a pre-determined threshold.

Certain embodiments may provide, for example, a method to provide patient compliance reporting to a healthcare agent. In certain embodiments, for example, the method may comprise downloading selected information from at least one HIPAA-compliant database. In certain embodiments, for example, the method may comprise pulling hashes and digital signatures applied to the hashes from a distributed ledger. In certain embodiments, for example, the method may comprise verifying the digital signatures, comprising: passing the hashes, the digital signatures, and public keys assigned to mobile medical devices through one or more digital signature verifying functions. In certain embodiments, for example, the method may comprise checking the accuracy of the downloaded selected information using the pulled hashes. In certain embodiments, for example, the method may comprise preparing de-identified patient compliance summaries, comprising: processing the selected information, wherein the de-identified patient compliance summaries may be indexed by the public keys. In certain embodiments, for example, the method may comprise communicating de-identified patient compliance summaries to at least one healthcare agent.

Certain embodiments may provide, for example, a method for managing patient compliance, comprising. In certain embodiments, for example, the method may comprise issuing mobile medical devices, with use instructions for treating medical conditions, to patients who have visited healthcare facilities, the medical devices comprising: processors, memories in communication with the processors, data sources in communication with the processors, reference to predetermined network coordinates stored in the memories, private digital signing keys, device identification codes assigned to the private digital signing keys, and program codes stored in the memories, the program codes executable by the processors to perform data communication operations, the data communication operations comprising: a) processing signals from the data sources to obtain device data; b) inserting dual payloads into network packets, first payloads of the dual payloads comprising device identification codes and at least portions of the device data, second payloads of the dual payloads comprising hashes of the at least portions of device data and device signatures applied to the hashes; and c) pushing the network packets to the predetermined network coordinates in response to internally-generated prompts. In certain embodiments, for example, the method may comprise modifying at least one course of treatment for at least one of the medical conditions, comprising: a) receiving at least one report, the at least one report comprising de-identified summaries of the at least portions of the device data; b) processing the at least one report to identify at least one patient having at least one health risk factor that is greater than at least one predetermined threshold; and c) modifying at least one course of treatment of the at least one patient.

Certain embodiments may provide, for example, a method to provide patient compliance reporting to a healthcare agent, comprising: i) downloading selected information from at least one HIPAA-compliant database; ii) pulling hashes and digital signatures applied to the hashes from a distributed ledger; iii) verifying the digital signatures, comprising: passing the hashes, the digital signatures, and public keys assigned to mobile medical devices through one or more digital signature verifying functions; iv) checking the accuracy of the downloaded selected information using the pulled hashes; v) preparing de-identified patient compliance summaries, comprising: processing the selected information, wherein the de-identified patient compliance summaries may be indexed by the public keys; and vi) communicating de-identified patient compliance summaries to at least one healthcare agent.

Certain embodiments may provide, for example, a method for managing patient compliance, comprising: i) issuing mobile medical devices, with use instructions for treating medical conditions, to patients who have visited healthcare facilities, the medical devices comprising: processors, memories in communication with the processors, data sources in communication with the processors, reference to predetermined network coordinates stored in the memories, private digital signing keys, device identification codes assigned to the private digital signing keys, and program codes stored in the memories, the program codes executable by the processors to perform data communication operations, the data communication operations comprising: a) processing signals from the data sources to obtain device data; b) inserting dual payloads into network packets, first payloads of the dual payloads comprising device identification codes and at least portions of the device data, second payloads of the dual payloads comprising hashes of the at least portions of device data and device signatures applied to the hashes; and c) pushing the network packets to the predetermined network coordinates in response to internally-generated prompts; and ii) modifying at least one course of treatment for at least one of the medical conditions, comprising: a) receiving at least one report, the at least one report comprising de-identified summaries of the at least portions of the device data; b) processing the at least one report to identify at least one patient having at least one health risk factor that is greater than at least one predetermined threshold; and c) modifying at least one course of treatment of the at least one patient.

Certain embodiments may provide, for example, a system for remote management of patient compliance. In certain embodiments, for example, the system may comprise a mobile medical device. In certain embodiments, for example, the mobile medical device may comprise a processor. In certain embodiments, for example, the mobile medical device may comprise a memory in communication with the processor. In certain embodiments, for example, the mobile medical device may comprise a data source in communication with the processor. In certain embodiments, for example, the mobile medical device may comprise a reference to predetermined network coordinates stored in the memory. In certain embodiments, for example, the mobile medical device may comprise program code stored in the memory, the program code executable by the processor to perform data communication operations, the data communication operations comprising: a) processing signals from the data source to obtain device data; b) generating digital signatures applied to the hashes, comprising: passing the hashes and a private key for the mobile medical device through one or more digital signing functions; c) inserting dual payloads into network packets, first payloads of the dual payloads comprising device identification codes for the mobile medical device and at least portions of the device data, second payloads of the dual payloads comprising hashes of the at least portions and the device signatures; and d) pushing the network packets to the predetermined network coordinates in response to internally-generated prompts.

In certain embodiments, for example, the data communication operations may further comprise: waiting for a message from a remote agent. In certain embodiments, for example, the data communication operations may further comprise: determining, based on receipt or non-receipt of the message, whether the network packets were successfully pushed. In certain embodiments, for example, the data communication operations may further comprise: deleting the dual payloads from the memory upon receipt of the message and determining the network packets were successfully pushed. In certain embodiments, for example, the data communication operations may further comprise: repushing the network packets to the predetermined network coordinates upon receiving the message or upon not receiving the message (for example upon not receiving the message after a predetermined duration of time).

In certain embodiments, for example, the mobile medical device may further comprise a user notification member in communication with the processor. In certain embodiments, for example, the user notification member may provide an indication of connectivity of the system to a wireless network. In certain embodiments, for example, the user notification member may provide an indication of success in the pushing. In certain embodiments, for example, the user notification member may provide an indication of failure in the pushing. In certain embodiments, for example, the user notification member may comprise a light source (for example a light emitting diode). In certain embodiments, for example, the user notification member may be configured to emit light in predetermined patterns to indicate one or more of connectivity to a wireless network, success in the pushing, and failure in the pushing.

A system for remote management of patient compliance, comprising: a mobile medical device, comprising: i) a processor; ii) a memory in communication with the processor; iii) a data source in communication with the processor; iv) a reference to predetermined network coordinates stored in the memory; and v) program code stored in the memory, the program code executable by the processor to perform data communication operations, the data communication operations comprising: a) processing signals from the data source to obtain device data; b) generating digital signatures applied to the hashes, comprising: passing the hashes and a private key for the mobile medical device through one or more digital signing functions; c) inserting dual payloads into network packets, first payloads of the dual payloads comprising device identification codes for the mobile medical device and at least portions of the device data, second payloads of the dual payloads comprising hashes of the at least portions and the device signatures; and d) pushing the network packets to the predetermined network coordinates in response to internally-generated prompts.

### BRIEF DESCRIPTION OF THE DRAWINGS

FIG. 1: A schematic illustration of processing a dual payload from a data source.

FIG. 2: A schematic illustration of mobile device registration.

FIG. 3: A schematic illustration remote patient compliance monitoring incorporating dual payload processing and a distributed ledger.

FIG. 4: A schematic illustration remote patient compliance monitoring incorporating dual payload processing, a distributed ledger and a compliance reporting service.

FIG. 5: A schematic illustration remote patient compliance monitoring.

FIG. 6: A schematic illustration remote patient compliance monitoring incorporating a distributed ledger.

FIG. 7: A schematic illustration remote patient compliance monitoring incorporating a distributed ledger and a compliance reporting service.

FIG. 8: Schematic view of a blockchain having a starting block and a series of transactional blocks.

FIG. 9: Schematic view of a blockchain being assembled.

FIG. 10: Schematic view of a distributed ledger ecosystem.

### DETAILED DESCRIPTION

In the description herein, the US Health Insurance Portability and Accountability Act (HIPAA)-compliant databases and frameworks are described as exemplary privacy-complant databases. However, the invention is not limited to HIPPA-compliant databases, but, rather, can be applied to any privacy-compliant databases, such as Europe's General Data Protection Regulations (GDPR), Japan's Act on the Protection of Personal In-formation (APPI), Canada's Personal Information Protection and Electronic Documents Act (PIPEDA), Austrailia's Privacy Act 1988, and the like.

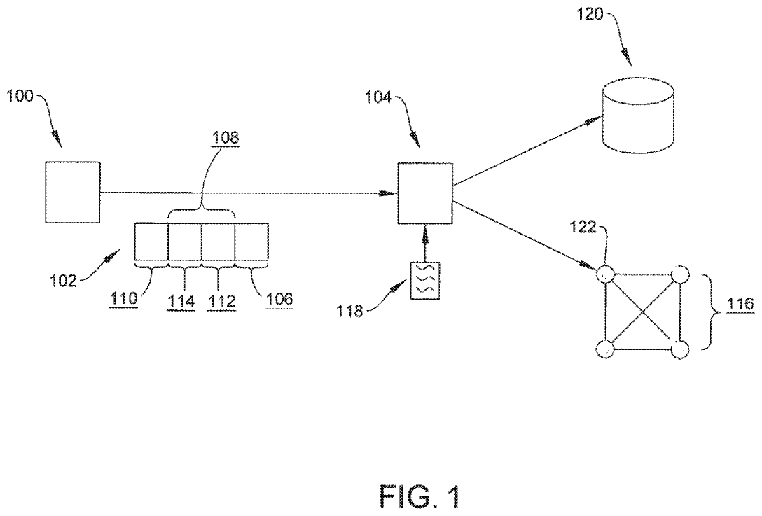

A schematic illustration of a framework (for example a HIPAA-compliant framework) for storing data and data provenance parameters is shown in FIG. 1. A data source 100 pushes a network packet 102 to a server 104, the network packet 102 comprising a header 106 with destination coordinates for the server 104, a dual payload 108 and a digital signature 110 applied to the dual payload 108. The dual payload 108 includes: a first payload 112 comprising de-identified (or anonymous or device-identified) data without any personal identification information, and a second payload 114 comprising a message containing a hash of the de-identified (or anonymous or device-identified) data, wherein the message is a set of instructions recognizable by a distributed ledger network 116 to encode the hash in a distributed ledger. Following receipt of the network packet 102 by the server 104, the server 104 verifies the digital signature by verifying that a public key for the data source is present in a proprietary list 118 of public keys assigned to authorized data sources, extracts the de-identified (or anonymous or device-identified) data and the message from the dual payload 108, inserts the de-identified (or anonymous or device-identified) data into a HIPAA-compliant database 120, and pushes the message (without any patient identification information) to a peer 122 of the distributed ledger network 116.

The data source 100 may be a mobile medical device, for example a hand-held nebulizer equipped with a cellular radio. The mobile medical device may comprise a processor, a memory, one or more private cryptographic keys, and necessary program code in order to compute the hash and the digital signature, and to form the network packet. The network packet 102 may traverse one or more of a cellular network, the public Internet, and an enterprise network to reach the server. The data source 100 and the server 104 may negotiate a network connection for communication of the network packet. The network connection may comprise one or more of a Transmission Control Protocol (TCP) connection, a User Datagram Protocol (UDP) connection, a Bluetooth connection, a Wi-Fi connection, a cellular network connection, or a connection according to a different network protocol. The network connection may be encrypted, for example the connection may be encrypted according to Transport Layer Security (TLS) protocol. The network packet 102 may comprise additional headers to enable transport according to one or more protocol for navigating one or more of a cellular network, the public Internet, and an enterprise network to reach the server. The proprietary list of public keys 116 may specify public keys for data sources that have been registered by one or more patients who have provided an executed HIPAA release form and a registration code for the data source 100. The message may contain a digital signature provided by the data source 100 and required by the distributed ledger network 116. One or more of the server 104, the database 120, the peer 122 of the distributed ledger network, and the entire distributed ledger network 116 may be contained in one or more enterprise networks (for example one enterprise network). The digital ledger network 116 may be a private distributed ledger. The digital ledger network 116 may be a public distributed ledger. The peer 122 may execute a smart contract in response to receiving the message.

FIG. 1 describes certain exemplary embodiments, but other variations fall within scope of the disclosure. In certain embodiments, for example, the server 104 may provide an additional digital signature to authorize the digital ledger network 116 to encode the hash. In certain embodiments, for example, the network packet 102 may be exclusive of the digital signature 110 applied to the dual payload 108 and the verification of the of the digital signature 110 may not be performed.

Certain embodiments (for example the embodiment disclosed in FIG. 1) may provide, for example, a method, a system, a product (for example a computer program product), a distributed system (for example a distributed computer program), software, middleware, computing infrastructure and/or apparatus (or a combination of two or more of the foregoing) comprising a mobile medical device. In certain embodiments, for example, the mobile medical device may be a nebulizer. In certain embodiments, for example, the nebulizer may be configured to nebulize a therapeutically effective unit dose of a medication (for example a medication for treatment of COPD or asthma). In certain embodiments, for example, the nebulizer may be an air-driven jet nebulizer (for example a nebulizer connected to an air compressor such as a Pari LC Jet Plus Nebulizer connected to a Pari Master or a Pari VIOS compressor). In certain embodiments, for example, the nebulizer may be a vibrating mesh nebulizer. In certain embodiments, for example, the vibrating mesh nebulizer may be handheld. In certain embodiments, for example, the vibrating mesh nebulizer may comprise a removable and/or disposable medicine cup. In certain embodiments, for example, the nebulizer may be a hand-held, battery-powered nebulizer (for example a hand-held, battery powered, vibrating mesh nebulizer). In certain embodiments, for example, the vibrating mesh nebulizer may nebulize all but no more than 0.05 mL of the sterile nebulization solution (for example, the vibrating mesh nebulizer may retain less than 0.05 mL residual sterile nebulization solution following administration of the therapeutically effective dosage regimen), for example all but no more than 0.02 mL of the sterile nebulization solution. In certain embodiments, for example, the nebulizer may be an ultrasonic nebulizer. In certain embodiments, for example, the nebulizer may be a breath-actuated nebulizer. In certain embodiments, for example, mobile medical device may inhaler. In certain embodiments, for example, the inhaler may be an actuated droplet inhaler (for example a Respimat inhaler). In certain further embodiments, for example, the inhaler may comprise a 4.5 mL plastic container crimped into an aluminum cylinder.

In certain embodiments, for example, the mobile medical device may be a continuous positive airway pressure (CPAP) device. In certain embodiments, for example, the mobile medical device may be a bilevel positive airway pressure device. In certain embodiments, for example, the mobile medical device may be a glucometer. In certain embodiments, for example, the mobile medical device may be a pulse oximeter. In certain embodiments, for example, the mobile medical device may be an oxygen delivery system. In certain embodiments, for example, the mobile medical device may be a blood pressure monitor/cuff.

Certain embodiments (for example the embodiment disclosed in FIG. 1) may provide, for example, a method, a system, a product (for example a computer program product), a distributed system (for example a distributed computer program), software, middleware, computing infrastructure and/or apparatus (or a combination of two or more of the foregoing) comprising a digital signature. In certain embodiments, for example, the digital signature for a message (for example a message comprising data) may be obtained as the output of a signing algorithm that has received the message and a cryptographic private key as inputs. In certain embodiments, for example, the message may be authenticated, or the digital signature may be verified, by verifying the digital signature applied to the message (for example by recovering the message from the digital signature (or, equivalently, by verifying the authenticity of the message) by inputting the message, the digital signature, and a cryptographic public key through one or more signature verifying algorithms). In certain embodiments, for example, the signing algorithm and the signature verifying algorithm may be selected from the group consisting of an RSA (Rivest-Shamir-Adleman)-based signature scheme (for example RSA-PSS (Probabilistic Signature Scheme), the Digital Signature Algorithm (DSA), the Elliptic Curve DSA (ECDSA), the Edwards-curve Digital Signature Algorithm, Ed25519, the ElGamal signature scheme, the Schnorr signature algorithm, the Pointcheval-Stern signature algorithm, the Rabin signature algorithm, a pairing-based algorithm (for example, the Boneh-Lynn-Shacham signature scheme), and an aggregate signature algorithm. In certain embodiments, for example, the cryptographic public key is generated with the cryptographic public key using a key generation algorithm.

Certain embodiments (for example the embodiment disclosed in FIG. 1) may provide, for example, a method, a system, a product (for example a computer program product), a distributed system (for example a distributed computer program), software, middleware, computing infrastructure and/or apparatus (or a combination of two or more of the foregoing) comprising a hash (or, equivalently, hashed value). In certain embodiments, for example, the hashed value may be obtained by passing data through a hashing algorithm. In certain embodiments, for example, the hashing algorithm may be selected from the group consisting of BLAKE-256, BLAKE-512, BLAKE2s, BLAKE2b, Elliptic Curve Only Hash (ECOH), the Fast Syndrome-based (FSB) hash, GOST, Grostl, HAS-160, HAVAL, JH, the Message Digent-2 (MD2) algorithm, MD4, MD5, MD6, RadioGatún, the RACE Integrity Primitives Evaluation Message Digest (RIPEMD), RIPEMD-128, RIPEMD-160, RIPEMD-320, the Secure Hash Algorithm-1 (SHA-1), SHA-2, SHA-224, SHA-256, SHA-384, SHA-512, SHA-3, Skein, Snefru, Spectral Hash, Streebog, SWIFFT, Tiger, Whirlpool-0, Whirlpool-T, and Whirlpool.

Certain embodiments (for example the embodiment disclosed in FIG. 1) may provide, for example, a method, a system, a product (for example a computer program product), a distributed system (for example a distributed computer program), software, middleware, computing infrastructure and/or apparatus (or a combination of two or more of the foregoing) comprising a distributed ledger, subcomponents and/or ancillary components of a distributed ledger, communications to/from a distributed ledger, smart contracts operating in an ecosystem comprising the distributed ledger, and/or optional distributed ledger applications (for example a wallet such as a mobile wallet). A distributed ledger is a distributed database that maintains information, e.g., a list of data records, computer program code (for example an executable program such as a smart contract), etc. In certain embodiments, for example, the information may comprise financial information, such as a designated portion of one or more financial accounts. The security of the information maintained within a distributed ledger may be enhanced by the fact that the ledger is distributed across plural nodes, which nodes may be one or more systems, machines, computers, databases, data stores or the like operably connected with one another. In certain embodiments, for example, each of the nodes or multiple nodes may be maintained by different entities. A distributed ledger may works without a central repository or single administrator. One well-known application of a distributed ledger is the public ledger of transactions for cryptocurrencies such as used in Bitcoin. In the case of Bitcoin, the data records recorded in the distributed ledger are enforced cryptographically and stored on the nodes of the distributed ledger.

In certain embodiments, for example, a distributed ledger may provide a number of advantages over traditional databases. In certain embodiments, for example, information may be added to the distributed ledger in blocks, wherein the blocks are added in an ordered sequence (or chain). In certain embodiments, for example, the chain of blocks (i.e., the blockchain) may be maintained to provide a history of the information added to the distributed ledger. In certain embodiments, for example, each added block may store a reproducible signature (for example a hash) indicative of a previous state of the blockchain, whereby any alteration of information in a previously-added block in the blockchain will be detected because the resulting signatures in subsequent blocks would not match previously stored signatures. In certain embodiments, for example, records in the distributed ledger may secure trusted data by hashing the data into an ongoing chain of hash-based proof-of-work, forming a record that cannot be changed without redoing the proof-of-work.

In certain embodiments, for example, plural nodes hosting the distributed ledger may reach a consensus regarding the validity of information maintained with a block of the blockchain, e.g., a transaction contained on a transaction ledger, financial information or the like. Additionally, when multiple versions of information exist on the ledger, multiple nodes can converge on the most up-to-date version of the transaction. For example, in the case of a virtual currency transaction, any node within the distributed ledger that creates a transaction can determine within a level of certainty whether the transaction can take place and become final by confirming that no conflicting transactions (i.e., the same currency unit has not already been spent) confirmed by the distributed ledger elsewhere.

In certain embodiments, for example, a distributed ledger may comprise two types of records. The first type of record is the information type, which consists of the actual data stored in the distributed ledger. The second type of record is the block type, which confirms when and in what sequence certain information became recorded as part of the distributed ledger. In certain embodiments, for example, information may be created by participants using the distributed ledger in the normal course of business, for example, when someone sends a resource to another person. In certain embodiments, for example, blocks are created by users known as “miners” who use specialized software/equipment to create blocks. In certain embodiments, for example, nodes in possession of a block of the distributed ledger may communicate with other nodes to validate added information based on a set of rules that are defined by the particular system implementing the distributed ledger. For example, in the case of financial information, such as cryptocurrencies or the like, valid information may be digitally signed, conducted from a valid digital wallet and, in some cases, meet other criteria.

In certain embodiments, for example, a distributed ledger may be decentralized—meaning that a distributed ledger is maintained on multiple nodes of the distributed ledger. One node in the distributed ledger may have a complete or partial copy of the entire ledger or set of information and/or blocks on the distributed ledger. Transactions may be initiated at a node of a distributed ledger and communicated to the various other nodes of the distributed ledger. Any of the nodes can validate information, add the information to its copy of the distributed ledger, and/or broadcast the information, its validation (in the form of a block) and/or other data to other nodes. This other data may include time-stamping, such as is used in financial resource distributed ledgers. Various other specific-purpose implementations of distributed ledgers have been developed. These include distributed domain name management, decentralized crowd-funding, synchronous/asynchronous communication, decentralized real-time ride sharing and even a general purpose deployment of decentralized applications.

In certain embodiments, for example, the distributed ledger may be public (for example a distributed ledger like Bitcoin, Litecoin, Ethereum, Zcash, Monero, Dash, Litecoin, Dodgecoin, or Hyperledger). In certain embodiments, for example, the distributed ledger may be private (for example a distributed ledger like Bankchain, MONAX, or Multichain). In certain embodiments, for example, the ledger may be permissionless (for example a ledger like blockchain). In certain embodiments, for example, the ledger may be permissioned (for example like Hyperledger, Kaleido, or PBFT). In certain embodiments, for example, the distributed ledger may be a consortium distributed ledger (for example a ledger like R3, EWF, B3i, or Corda).

In certain embodiments, for example, the distributed ledger may be an open-source distributed computing platform. In certain embodiments, for example, the distributed ledger may be a blockchain-based distributed computing platform and operating system. In certain embodiments, for example, the distributed ledger may utilize scripting functionality. In certain embodiments, for example, the distributed ledger may utilize smart contract functionality. In certain embodiments, for example, the distributed ledger may support a modified version of Nakamoto consensus via transaction-based state. In certain embodiments, for example, the distributed ledger may be an open-source, public, blockchain-based distributed computing platform and operating system utilizing smart contract functionality, and support a modified version of Nakamoto consensus via transaction-based state transitions (for example Ethereum).

Certain embodiments (for example the embodiment disclosed in FIG. 1) may provide, for example, a method, a system, a product (for example a computer program product), a distributed system (for example a distributed computer program), software, middleware, computing infrastructure and/or apparatus (or a combination of two or more of the foregoing) comprising a database. In certain embodiments, for example, the database may be a relational database. In certain embodiments, for example, the database may be an Oracle database. In certain embodiments, for example, the database may be a MySQL database. In certain embodiments, for example, the database may be a Microsoft SQLServer database. In certain embodiments, for example, the database may be a PostgreSQL database. In certain embodiments, for example, the database may be a DB2 database. In certain embodiments, for example, the database may be a non-relational database. In certain embodiments, for example, the database may be a key-value store (for example Redis or Amazon DynamoDB). In certain embodiments, for example, the database may be a wide column store (for example Cassandra, Scylla, or HBase). In certain embodiments, for example, the database may be a document store (for example MongoDB or Couchbase). In certain embodiments, for example, the database may be a graph database (for example Neo4J or Datastax Enterprise Graph). In certain embodiments, for example, the database may comprise a search engine (for example Elasticsearch, Splunk, or Solr). In certain embodiments, for example, the data source (for example a mobile medical device) may comprise one of the foregoing types of databases in the memory to store the generated data.

Certain embodiments (for example the embodiment disclosed in FIG. 1) may provide, for example, a method, a system, a product (for example a computer program product), a distributed system (for example a distributed computer program), software, middleware, computing infrastructure and/or apparatus (or a combination of two or more of the foregoing) comprising a network connection. In certain embodiments, for example, the network connection may be configured according to X10, Ethernet, RS-485, 6LoWPAN, Bluetooth LE (BLE), ZigBee, Z-Wave, or two or more of the foregoing protocol. In certain embodiments, for example, the network connection may utilize a cellular protocol, for example 3G, 4G, 4GLTE, 5G, or 6G.

Certain embodiments may provide, for example, a method, a system, a product (for example a computer program product), a distributed system (for example a distributed computer program), software, middleware, computing infrastructure and/or apparatus (or a combination of two or more of the foregoing) comprising a data protocol used in transmission of data. In certain embodiments, for example, the data protocol may be used in transmission of data (for example device data, hash data, or messages configured for delivery to a distributed ledger network) from a data source (for example a mobile medical device) to a server. In certain embodiments, for example, the data protocol may be used in transmission of data (for example device data) from the server to a database. In certain embodiments, for example, the data protocol may be used in transmission of data (for messages configured for delivery to a distributed ledger network) from a server to a peer in a distributed ledger network. In certain embodiments, for example, the data protocol may be a machine-to-machine protocol. In certain embodiments, for example, the data protocol may be an Internet of Things (IoT) protocol. In certain embodiments, for example, the data protocol may comprise an MQ Telemetry Transport (MQTT) protocol. In certain embodiments, for example, the data protocol may comprise an Advanced Message Queuing Protocol (AMQP). In certain embodiments, for example, the data protocol may comprise a Simple/Streaming Text Oriented Messaging Protocol (STOMP). In certain embodiments, for example, the data protocol may comprise a Data Distribution Service DDS. In certain embodiments, for example, the data protocol may comprise a Constrained Application Protocol (CoAP). In certain embodiments, for example, the data protocol may comprise an Open Platform Communications Unified Architecture (OPC UA) protocol. In certain embodiments, for example, the data protocol may comprise a Java Message Service (JMS) protocol. In certain embodiments, for example, the data protocol may comprise an eXtensible Messaging and Presence Protocol (XMPP). In certain embodiments, for example, the data protocol may comprise a Representational State Transfer (REST) protocol. In certain embodiments, for example, the data protocol may comprise an Open Mobile Alliance Light Weight Machine-to-Machine (OMA LWM2M) protocol. In certain embodiments, for example, the data protocol may comprise a JavaScript Object Notation (JSON) protocol. In certain embodiments, for example, the data protocol may comprise a Simple Network Management Protocol (SNMP). In certain embodiments, for example, the data protocol may comprise a protocol conforming to Technical Report 069: CPE WAN Management Protocol (TR-069-CWMP). In certain embodiments, for example, the data protocol may conform to a Health Level 7 (HL7) specification. In certain embodiments, for example, the data protocol may conform to a Fast Healthcare Interoperability Resources (FHIR) specification. In certain embodiments, for example, the data protocol may conform to Continua Design Guidelines (CDG). In certain embodiments, for example, the data protocol may comprise Hypertext Transfer Protocol (HTTP). In certain embodiments, for example, the data protocol may conform to the Alljoyn framework. In certain embodiments, for example, the data protocol may comprise Modbus protocol (for example Modbus over TCP and UDP). In certain embodiments, for example, the data protocol may conform to VITA 49 radio transport packet specification. In certain embodiments, for example, the data protocol may conform to Edgent protocol. In certain embodiments, for example, the data protocol may comprise a file transfer protocol. In certain embodiments, for example, the data protocol may comprise a domain name server protocol. In certain embodiments, for example, the data protocol may comprise an Internet Control Message Protocol (ICMP). In certain embodiments, for example, the data protocol may comprise a structured query language protocol. In certain embodiments, for example, the data protocol may comprise a publish-subscribe messaging pattern protocol. In certain embodiments, for example, the data protocol may comprise a data distribution service protocol. In certain embodiments, for example, the data protocol may comprise a data structure identifier. In certain embodiments, for example, the data protocol may comprise a data topic. In certain embodiments, for example, the data protocol may comprise a data type (for example “string”, “integer”, “unsigned integer”, “Boolean”, “floating point”, “double precision”, etc.). In certain embodiments, for example, the data protocol may indicate an allowed range (for example a continuous range or a list of allowed values) of values for a data payload. In certain embodiments, for example, the data protocol may comprise a data definition identifier.

Certain embodiments may provide, for example, a method, a system, a product (for example a computer program product), a distributed system (for example a distributed computer program), software, middleware, computing infrastructure and/or apparatus (or a combination of two or more of the foregoing) comprising a command syntax, command type, and/or command type identifier accompanying transmitted data (for example a packet of data sent to a database may be accompanied by a command instructing the database how to format or operate on the data). In certain embodiments, for example, the command type may comprise a SQL command and/or statement, for example the command type may comprise SQLread, SQLwrite, AND/OR, ALTER TABLE, AS (alias), BETWEEN, CREATE DATABASE, CREATE TABLE, CREATE INDEX, CREATE VIEW, DELETE, DROP DATABASE, DROP INDEX, DROP TABLE, EXISTS, GROUP BY, HAVING, IN, INSERT INTO, INNER JOIN, LEFT JOIN, RIGHT JOIN, FULL JOIN, LIKE, ORDER BY, SELECT, SELECT *, SELECT DISTINCT, SELECT INTO, SELECT TOP, TRUNCATE TABLE, UNION, UNION ALL, UPDATE, WHERE, or a combination of two or more of the foregoing commands. In certain embodiments, for example, the command type may comprise a DNS command, for example the command type may comprise ipconfig, trace route, netstat, arp, route, hostname, control netconnections, or a combination of two or more of the foregoing commands. In certain embodiments, for example, the command type may comprise an FTP command, for example the command type may comprise !, $, ?, account, append, ascii, beep, binary, bye, case, cd, cdup, chmod, close, cr, debug, delete, dir, disconnect, exit, form, get, glob, hash, help, idle, image, ipany, ipv4, ipv6, lcd, ls, macdef, mdelete, mdir, mget, mkdir, mls, mode, modtime, mput, newer, nlist, nmap, ntrans, open, passive, prompt, proxy, put, pwd, qc, quit, quote, recv, reget, rename, reset, restart, rhelp, rmdir, rstatus, runique, send, sendport, site, size, status, struct, sunique, system, tenex, tick, trace, type, umask, user, verbose, or a combination of two or more of the foregoing commands. In certain embodiments, for example, the command type may comprise a Telnet, an Rlogin, an Rsh, or a Secure Shell command. In certain embodiments, for example, the command type may comprise an ICMP command, for example the command type may comprise PING, TRACEROUTE, ICMP PERMIT, ICMP DENY, or a combination of two or more of the foregoing commands. In certain embodiments, for example, the command type may comprise an MQTT command. In certain embodiments, for example, the command type may be a function call. In certain embodiments, for example, the function call may be a call to or from a smart contract. In certain embodiments, for example, the function call may be signed.

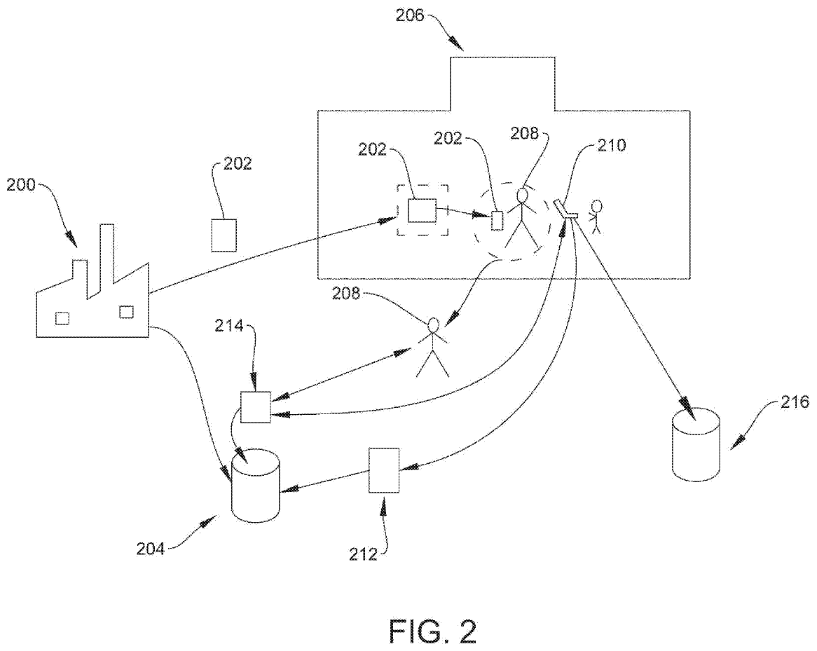

A schematic illustration of an approach to device registration to support subsequent data collection for use compliance monitoring is shown in FIG. 2. A mobile device manufacturer 200 manufactures a mobile device 202 and pre-populates an identification code for the device 202 in a device database 204, and ships the device 202 which ends up in inventory (shown by dashed lines) at a healthcare facility 206. At a future point in time, the device 202 is assigned to a patient 208, and the healthcare facility 206 assists the patient 208 to register the device 202 with the manufacturer by a computer 210 accessing a first Internet portal 212. Registration includes providing patient identification and contact information, a device registration code which references the device identification code, and a HIPAA release form executed by the patient 208. The device registration code may be located on the device 202, for example on a sticker affixed to the outside of the device 202. The registration code may be scannable by a digital scanner connected (not shown) to the computer 210. In a further action, the healthcare facility 206 may submit a request via the computer 210 (or another computer) to a second Internet portal 214 for access to data generated by the device 202, and the request will be submitted to the patient 208 for approval. The communication to the patient 208 and the approval by the patient 208 to grant access (or refusal to grant access) may be electronic (for example a text message or an email) If the request is approved, access credentials are sent from the second Internet portal 214 to the computer 210 and the second Internet portal 214 updates the device database 204 with the credentials. A record may be written to the smart contract indicating grant, refusal, or revocation of the access. The computer 210 sends instructions to append the identification code for the device 202, identification information for the patient, and patient-specific operating instructions for the device 202 to a HIPPA-compliant electronic health record database 216. The patient leaves the healthcare facility 206 with the mobile device 202.

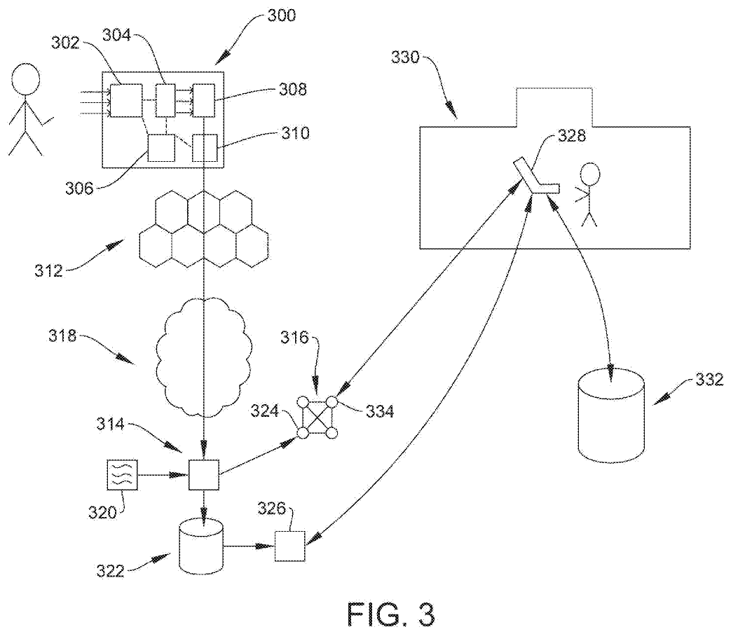

A schematic illustration of an approach to remote patient compliance monitoring incorporating a distributed ledger is depicted in FIG. 3. An operator (for example an intended patient who has been assigned a mobile device) activates a mobile device 300 for a period of time on each of 3 separate occasions (the number of occasions is dictated by operating instructions provided to an intended patient and the number “3” is selected for illustration purposes only—any number of occasions, including one occasion or more than three occasions, is contemplated herein) by engaging an activation component 302 (for example a button) on the device 300. In response, on each of the 3 occasions an operating component 304 of the device 300 generates operating data and a processor 306 of the device 300 executes instructions to cause the operating data to be stored in a memory 308 of the device 300. Next, a cellular radio 310 of the device 300 detects a cellular network 312 and, following detection of the cellular network 312, the processor 306 executes instructions to form and transmit a network packet. The formed network packet comprises a header with destination coordinates for a first server 314 and a dual payload. The dual payload includes: a first payload comprising at least a portion of the stored operating data and a device identification code, and a second payload comprising a message containing a hash of the at least a portion of the stored operating data, and a digital signature of the device 300 applied to the hash, wherein the message is a set of instructions recognizable by a distributed ledger network 316 to encode the hash in a distributed ledger. The network packet is transferred via the cellular radio 310 to the cellular network 312. After traversing a portion of the cellular network the network packet is passed to a packet-switched network 318 (for example the public Internet) and routed to the first server 314. Following receipt of the network packet by the first server 314, first server 314 verifies the digital signature by passing a public key associated with the device identification code (for example the public key may be the device identification code), the digital signature, and the hash through one or more signature verifying functions, verifying that the public key is present in a proprietary list 320 of public keys assigned to authorized devices, extracts the operating data and the message from the dual payload, inserts the operating data into a HIPAA-compliant database 322, and pushes the message (without any patient identification information) to a peer 324 of the distributed ledger network 316. The processor 306 may execute instructions to encrypt the operating data before the operating data is stored in the memory 308, or the processor 306 may execute instructions to encrypt the operating data before it is transmitted to the cellular network 312. Communications via the cellular network and the packet-switched network may be encrypted or otherwise secured (for example by transport layer security between the device 300 and the first server 314.

The distributed ledger 316 may be a private ledger or a public ledger such as Ethereum configured to provide an immutable record of the time series data by storing a reference to the addition of the hash to an immutability construct such as a block in a blockchain The first server 314 may be a secure server that is purpose-configured to receive data exclusively from predetermined devices based on a pre-populated list of device identification codes and/or proprietary list 320 of public keys, with write access to the device database 322 otherwise strictly limited to predetermined administrative functions (for example, pre-populating device identification codes), and data export from the device database 322 may be limited to predefined internal report generation and requests from authorized third parties via a second server 326.

Remote compliance monitoring of an intended patient's use of the mobile device 300 comprises (a) using a compliance device 328 (for example a computer at a healthcare facility 330) to obtain the device identification code and access credentials for a patient from a HIPPA-compliant EHR database 332, (b) supplying a data request with access credentials to the second server 326 to obtain time series of operating data, (c) verifying access (for example by a smart contract) by confirming the access credentials are present on a list of valid access credentials, and (d) obtaining one or more hashes of the time series of operating data from a peer 334 (for example a peer assigned to the healthcare facility 330) in the distributed ledger network 316. The time series of operating data are run through a hash function on the compliance device 328 and the results are confirmed to match the one or more hashes obtained from the distributed ledger network 316. Upon confirmation, the compliance device 328 compares the time series of operating data with the operating instructions for the intended user and determines whether the course of use of the mobile device 300 complies with the instructions. If the use is non-compliant and a risk is detected that exceeds a predetermined threshold, remote compliance monitoring may trigger communication with the intended patient, for example to provide updated instructions for operating the device 300.

FIG. 3 describes certain exemplary embodiments, but other variations fall within scope of the disclosure. In certain embodiments, for example, the remote compliance monitoring may be performed at a different location from the healthcare facility 330. Certain embodiments, for example, may combine portions or all of FIG. 1, FIG. 2, and/or FIG. 3.

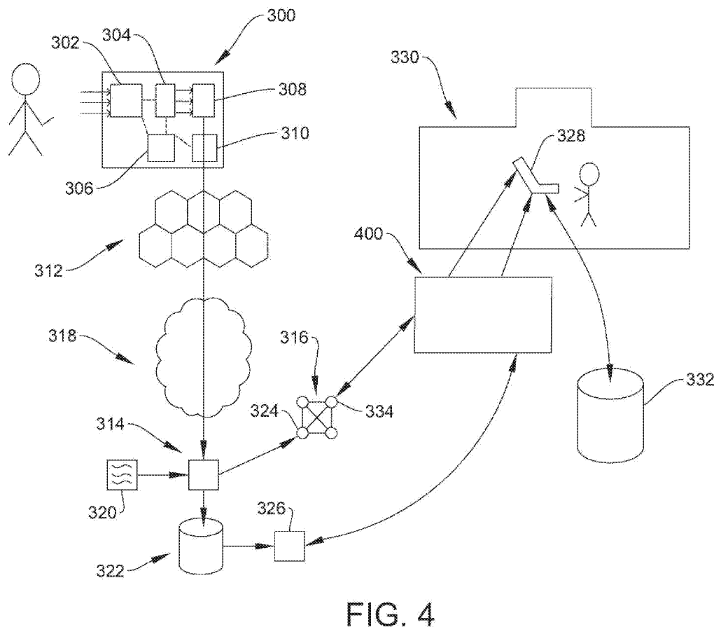

A schematic illustration of an approach to remote patient compliance monitoring incorporating a distributed ledger and a compliance reporting service is depicted in FIG. 4. An operator (for example an intended patient who has been assigned a mobile device) activates a mobile device 300 for a period of time on each of 3 separate occasions (the number of occasions is dictated by operating instructions provided to an intended patient and the number “3” is selected for illustration purposes only—any number of occasions, including one occasion or more than three occasions, is contemplated herein) by engaging an activation component 302 (for example a button) on the device 300. In response, on each of the 3 occasions an operating component 304 of the device 300 generates operating data and a processor 306 of the device 300 executes instructions to cause the operating data to be stored in a memory 308 of the device 300. Next, a cellular radio 310 of the device 300 detects a cellular network 312 and, following detection of the cellular network 312, the processor 306 executes instructions to form and transmit a network packet. The formed network packet comprises a header with destination coordinates for a first server 314 and a dual payload. The dual payload includes: a first payload comprising at least a portion of the stored operating data and a device identification code, and a second payload comprising a message containing a hash of the at least a portion of the stored operating data, and a digital signature of the device 300 applied to the hash, wherein the message is a set of instructions recognizable by a distributed ledger network 316 to encode the hash in a distributed ledger. The network packet is transferred via the cellular radio 310 to the cellular network 312. After traversing a portion of the cellular network the network packet is passed to a packet-switched network 318 (for example the public Internet) and routed to the first server 314. Following receipt of the network packet by the first server 314, first server 314 verifies the digital signature by passing a public key associated with the device identification code (for example the public key may be the device identification code), the digital signature, and the hash through one or more signature verifying functions, verifying that the public key is present in a proprietary list 320 of public keys assigned to authorized devices, extracts the operating data and the message from the dual payload, inserts the operating data into a HIPAA-compliant database 322, and pushes the message (without any patient identification information) to a peer 324 of the distributed ledger network 316. The processor 306 may execute instructions to encrypt the operating data before the operating data is stored in the memory 308, or the processor 306 may execute instructions to encrypt the operating data before it is transmitted to the cellular network 312.

Remote compliance monitoring of an intended patient's use of the mobile device 300 comprises a compliance service 400 (a) using a compliance device 328 (for example a computer at a healthcare facility 330) to obtain the device identification code and access credentials for a patient from a HIPPA-compliant EHR database 332, (b) supplying a data request with access credentials to the second server 326 to obtain the time series of operating data, (c) verifying access (for example by a smart contract) by confirming the access credentials are present on a list of valid access credentials, and (d) obtaining the recorded hash of the operating data from a peer 334 (for example a peer assigned to the healthcare facility 330) in the distributed ledger network 316. The time series is run through a hash function on the compliance device 328 and the result is confirmed to match the hash obtained from the distributed ledger network 316. Upon confirmation, the compliance service 400 compares the time series of operating data with the operating instructions for the intended user and generates a report which is transmitted to the compliance device 328 where it is utilized to perform the remote compliance monitoring to determine whether the course of use of the mobile device 300 complies with the instructions. If the use is non-compliant (for example the use comprises over-use or underuse) and a risk is detected that exceeds a predetermined threshold (for example over-use indicates a risk of disease deterioration), remote compliance monitoring may trigger communication with the intended patient, for example to provide updated instructions for operating the device 300.

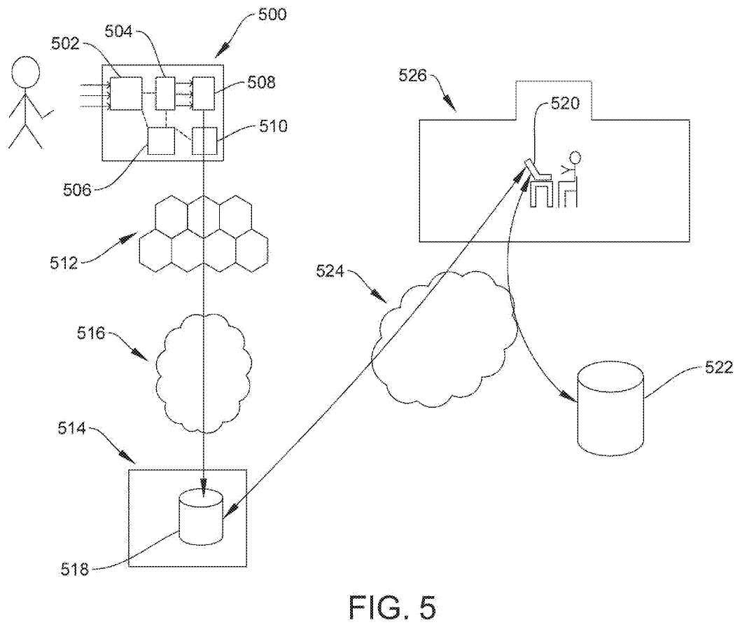

A schematic illustration of an approach to remote patient compliance monitoring is depicted in FIG. 5. An operator (for example an intended patient who has been assigned a mobile device) activates a mobile device 500 for a period of time on each of 3 separate occasions (the number of occasions is dictated by operating instructions provided to an intended patient and the number “3” is selected for illustration purposes only—any number of occasions, including one occasion or more than three occasions, is contemplated herein) by engaging an activation component 502 (for example a button) on the device 500. In response, on each of the 3 occasions an operating component 504 of the device 500 generates operating data and a processor 506 of the device 500 executes instructions to cause the operating data to be stored in a memory 508 of the device 500. Next, a cellular radio 510 of the device 500 detects a cellular network 512 and, following detection of the cellular network 512, the processor 506 executes instructions to cause the operating data to be copied from the memory 508, placed—along with a preconfigured identification code for the device and a preconfigured destination address associated with a server hardware 514—into a network packet, and transferred via the cellular radio to the cellular network 512. After traversing a portion of the cellular network the network packet is passed to a packet-switched network 516 (for example the public Internet) and routed to the server hardware. The operating data and identification code for the mobile device 500 are extracted from the packet and the operating data is encoded as time series of operating data (indexed at least in part by the identification code for the mobile device 500) in a device database 518 resident on the server hardware 514. The device database 518 does not contain any personal identification information for an intended user of the device 500. The server hardware 514 may be a secure server hardware that is purpose-configured to receive data exclusively from predetermined devices based on a pre-populated list of device identification codes, and write access to the device database 518 may otherwise be strictly limited to predetermined administrative functions (for example, pre-populating device identification codes). Optionally, the processor 506 may execute instructions to encrypt the operating data before the operating data is stored in the memory 508, or the processor 506 may execute instructions to encrypt the operating data before it is transmitted to the cellular network 512.

Subsequently, remote compliance monitoring of an intended patient's use of the mobile device 500 comprises a compliance device 520 obtaining the identification code for the device 500 and operating instructions for the device 500 (for example a doctor's prescription) from a HIPPA-compliant EHR database 522 via a network 524, and uses the identification code for the device 500 as an index to retrieve the time series of operating data from the device database 518 via the network 524. The remote compliance monitoring compares the time series of operating data with the operating instructions for the intended user and determines whether the course of use of the mobile device 500 complies with the instructions. If the use is non-compliant and a risk is detected that exceeds a predetermined threshold, remote compliance monitoring may trigger communication with the intended patient, for example to provide updated instructions for operating the device 500.

FIG. 5 describes certain exemplary embodiments, but other variations fall within scope of the disclosure. In certain embodiments, for example, the remote compliance monitoring may be performed at a healthcare facility 526 or at a location different location from the healthcare facility 526. In certain embodiments, for example, the network 524 may overlap the network 516, and/or the data from the EHR database 522 may be received by a different network than the time series of operating data. Certain embodiments, for example, may combine portions or all of FIGS. 1-5.

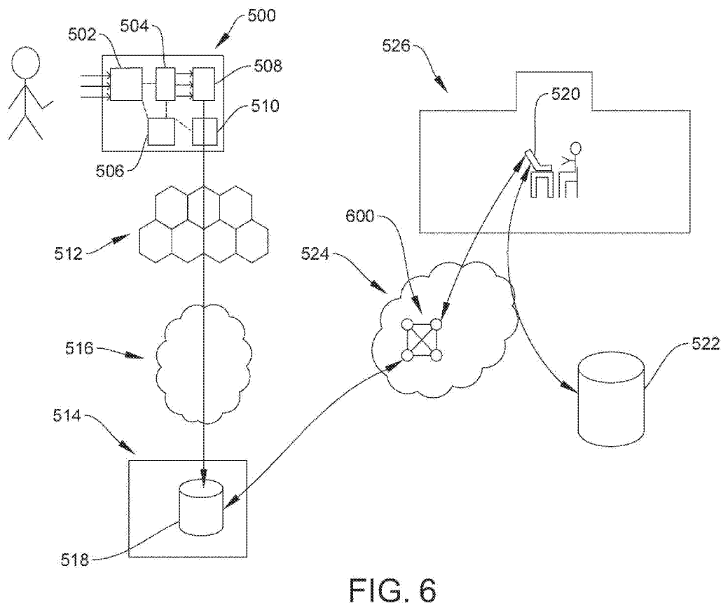

A schematic illustration of an approach to remote patient compliance monitoring incorporating a distributed ledger is depicted in FIG. 6. An operator (for example an intended patient who has been assigned a mobile device) activates a mobile device 500 for a period of time on each of 3 separate occasions (the number of occasions is dictated by operating instructions provided to an intended patient and the number “3” is selected for illustration purposes only—any number of occasions, including one occasion or more than three occasions, is contemplated herein) by engaging an activation component 502 (for example a button) on the device 500. In response, on each of the 3 occasions an operating component 504 of the device 500 generates operating data and a processor 506 of the device 500 executes instructions to cause the operating data to be stored in a memory 508 of the device 500. Next, a cellular radio 510 of the device 500 detects a cellular network 210 and, following detection of the cellular network 512, the processor 506 executes instructions to cause the operating data to be copied from the memory 508, placed—along with a preconfigured identification code for the device and a preconfigured destination address associated with a server 514—into a network packet, and transferred via the cellular radio to the cellular network 512. After traversing a portion of the cellular network the network packet is passed to a packet-switched network 516 (for example the public Internet) and routed to the server. The operating data and identification code for the mobile device 500 are extracted from the packet and the operating data is encoded as time series of operating data (indexed at least in part by the identification code for the mobile device 500) in a device database 518 resident on the server 514. The device database 500 does not contain any personal identification information for an intended user of the device 500. Optionally, the processor 506 may execute instructions to encrypt the operating data before the operating data is stored in the memory 508, or the processor 506 may execute instructions to encrypt the operating data before it is transmitted to the cellular network 512.

Subsequently, the time series of operating data (or one or more hashes derived from the time series of operating data) is published to a distributed ledger 600 via a network 524. The distributed ledger 600 may be a private ledger or a public ledger such as Ethereum configured to provide an immutable and optionally encrypted) record of the time series data. The server 514 may be a secure server that is purpose-configured to receive data exclusively from predetermined devices based on a pre-populated list of device identification codes, with write access to the device database 518 otherwise be strictly limited to predetermined administrative functions (for example, pre-populating device identification codes), and data export from the device database 518 may be limited to the publication to the distributed ledger 600 (e.g., according to a predefined protocol such as pre-approved JSON messages) and to predefined internal report generation.

Remote compliance monitoring of an intended patient's use of the mobile device 500 comprises a compliance device 520 obtaining the identification code for the device 500 and operating instructions for the device 500 (for example a doctor's prescription) from a HIPPA-compliant EHR database 522 via the network 524, and uses the identification code for the device 500 as an index to retrieve the time series of operating data (and/or one or more hashes (for example one hash for each data transmission from the mobile device) derived from the time series of operating data) from the distributed ledger 600 via the network 524. The one or more hashes may be used to generate a request for one or more dates associated with the time series. The remote compliance monitoring compares the time series of operating data with the operating instructions for the intended user and determines whether the course of use of the mobile device 500 complies with the instructions. If the use is non-compliant and a risk is detected that exceeds a predetermined threshold, remote compliance monitoring may trigger communication with the intended patient, for example to provide updated instructions for operating the device 500.

FIG. 6 describes certain exemplary embodiments, but other variations fall within scope of the disclosure. In certain embodiments, for example, the remote compliance monitoring may be performed at a different location from the healthcare facility 526. In certain embodiments, for example, the network 524 may overlap the network 516, and/or the data from the EHR database 522 may be received by a different network than the time series of operating data. Certain embodiments, for example, may combine portions or all of FIGS. 1-6.

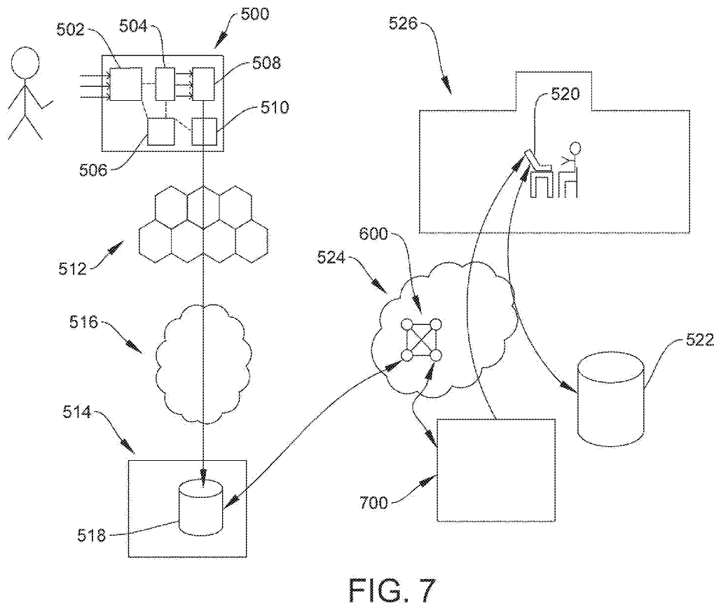

A schematic illustration of an approach to remote patient compliance monitoring incorporating a distributed ledger and a compliance reporting service is depicted in FIG. 7. An operator (for example an intended patient who has been assigned a mobile device) activates a mobile device 500 for a period of time on each of 3 separate occasions (the number of occasions is dictated by operating instructions provided to an intended patient and the number “3” is selected for illustration purposes only—any number of occasions, including one occasion or more than three occasions, is contemplated herein) by engaging an activation component 502 (for example a button) on the device 500. In response, on each of the 3 occasions an operating component 504 of the device 500 generates operating data and a processor 506 of the device 500 executes instructions to cause the operating data to be stored in a memory 508 of the device 500. Next, a cellular radio 510 of the device 500 detects a cellular network 512 and, following detection of the cellular network 512, the processor 506 executes instructions to cause the operating data to be copied from the memory 508, placed—along with a preconfigured identification code for the device and a preconfigured destination address associated with a server 514—into a network packet, and transferred via the cellular radio to the cellular network 512. After traversing a portion of the cellular network the network packet is passed to a packet-switched network 516 (for example the public Internet) and routed to the server. The operating data and identification code for the mobile device 500 are extracted from the packet and the operating data is encoded as time series of operating data (indexed at least in part by the identification code for the mobile device 500) in a device database 518 resident on the server 514. The device database 518 does not contain any personal identification information for an intended user of the device 500. Optionally, the processor 506 may execute instructions to encrypt the operating data before the operating data is stored in the memory 508, or the processor 506 may execute instructions to encrypt the operating data before it is transmitted to the cellular network 520.

Subsequently, the time series of operating data is published to a distributed ledger 600 via the network 524. The distributed ledger 600 may be a private ledger or a public ledger such as Ethereum configured to provide an immutable and optionally encrypted) record of the time series data. The server 514 may be a secure server that is purpose-configured to receive data exclusively from predetermined devices based on a pre-populated list of device identification codes, with write access to the device database 518 otherwise be strictly limited to predetermined administrative functions (for example, pre-populating device identification codes), and data export from the device database 518 may be limited to the publication to the distributed ledger 600 (e.g., according to a predefined protocol such as pre-approved JSON messages) and to predefined internal report generation.

Remote compliance monitoring of an intended patient's use of the mobile device 500 comprises a compliance service 700 obtaining the identification code for the device 500 and operating instructions for the device 500 (for example a doctor's prescription) from a HIPPA-compliant EHR database 522 via a network 524, and uses the identification code for the device 500 as an index to retrieve the time series of operating data from the distributed ledger 600 via the network 524. The compliance service 700 compares the time series of operating data with the operating instructions for the intended user and generates a report which is transmitted to a compliance device 520 utilized to perform the remote compliance monitoring to determine whether the course of use of the mobile device 500 complies with the instructions. If the use is non-compliant and a risk is detected that exceeds a predetermined threshold, remote compliance monitoring may trigger communication with the intended patient, for example to provide updated instructions for operating the device 500.

FIG. 7 describes certain exemplary embodiments, but other variations fall within scope of the disclosure. In certain embodiments, for example, the remote compliance monitoring may be performed at a different location from the healthcare facility 526. In certain embodiments, for example, the network 524 may overlap the network 516, and/or the data from the EHR database 522 may be received by a different network than the time series of operating data. Certain embodiments, for example, may combine portions or all of FIGS. 1-7.

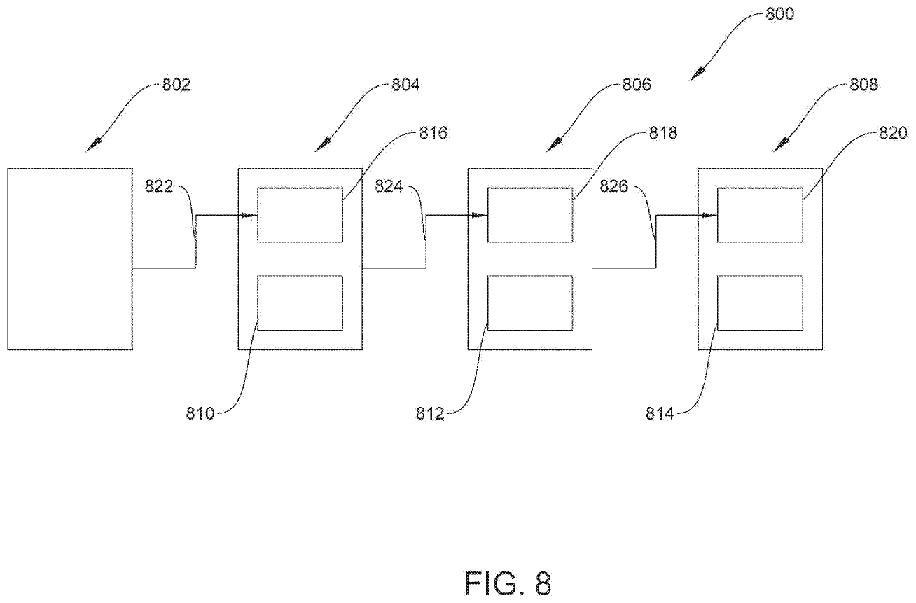

FIG. 8 schematically depicts a blockchain 800 having a starting block 802 (the so-called “genesis block”) and a subsequent series of transactional blocks (804, 806, and 808). Each transactional block (804, 806, or 808) comprises a transaction (810, 812, or 814, respectively) (for example the transfer of property from one party to another) and a hash value (816, 818, or 820, respectively), the hash value referencing the previous block. The hash value is a piece of ciphertext generated by passing (822, 824, or 826) at least a portion of the text of the previous block through at least one mathematical hash function. The hash values (816, 818, and 820) ensure that a block cannot be altered without causing a mismatch between the contents of the block and the hash values in all subsequent blocks. Moreover, any mismatch should be easy to detect because the at least one mathematical hash function is selected to ensure that even a small change to the content of a block produces a large change in the hash value generated from it. Of note, the hash values make it simple to track and audit the validity of the data, making blockchains much more difficult to hack or falsify.

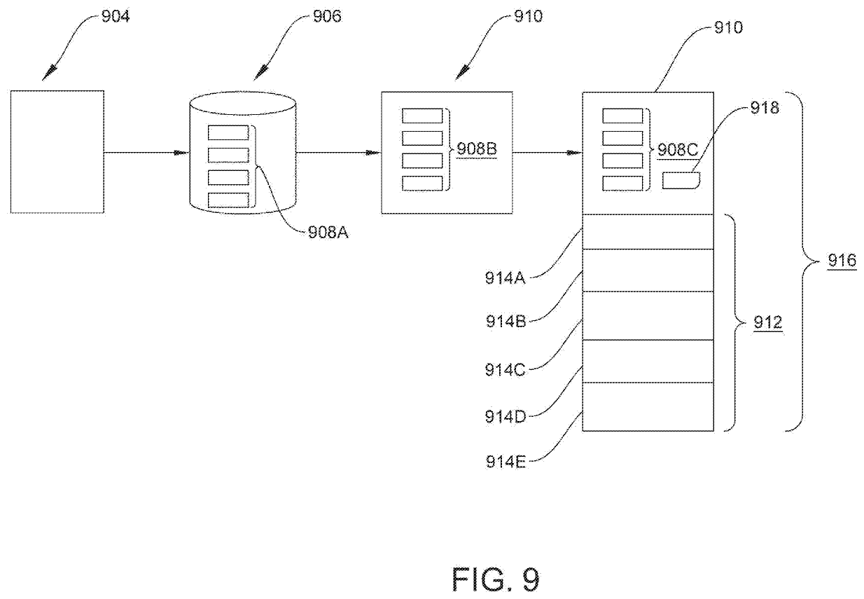

FIG. 9 is a schematic depiction of exemplary steps required to add transactions to a blockchain A wallet 904 transmits packet data containing a new transaction to one or more network participants 906 in a blockchain network, each of whom maintains their own copy of a list of new transactions 908A (so-called “unconfirmed transactions”). Each participant 906 may verify the veracity of the new transactions 908A (including verification of digital signatures associated with the new transactions) according to a pre-determined set of rules, either in partnership or in competition with other participants, and place the verified new transactions in a new block 910. The new block 910 may be appended to a prior instance of the blockchain 912 containing previously added blocks 914A-E as shown, and the provenance and immutability of the resulting blockchain 916 may be established by including a ciphertext signature 918 in the new block 910, wherein the ciphertext signature 918 is obtained by applying a cipher function (for example a hash or trap door function) constructively to all of the prior instance of the blockchain 912. The participant distributes the resulting blockchain 916 to the other network participants, and the participants apply consensus rules to either accept or reject the blockchain 916. If the blockchain 916 is accepted, it replaces the prior instance of the blockchain 912 and becomes the starting point for appending future blocks.

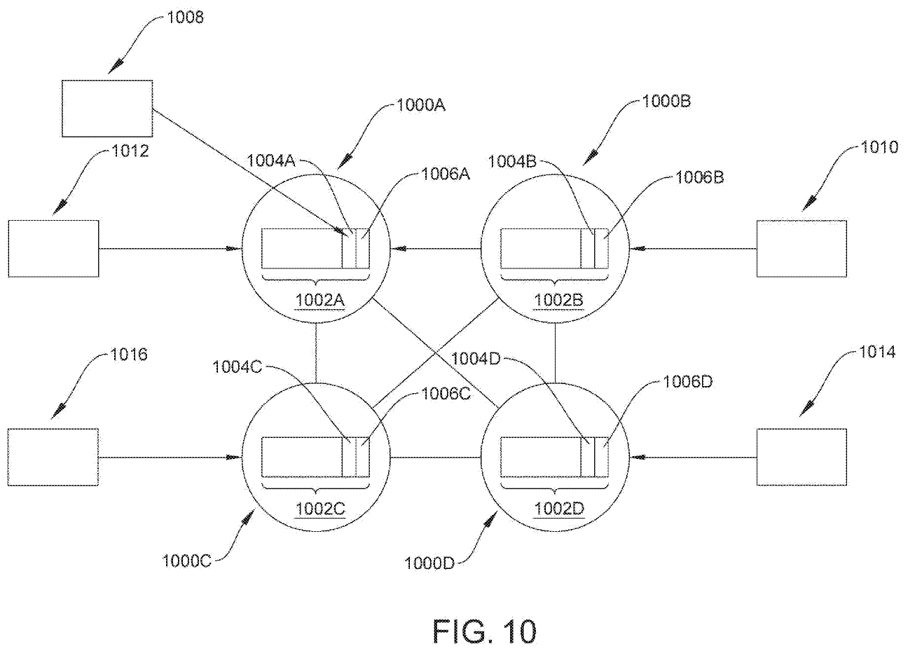

FIG. 10 is a schematic depiction of a distributed ledger ecosystem configured to process user-originated transactions and smart contracts. Consensus nodes 1000A-D communicate among one another to verify transactions, authorize messages that trigger execution of smart contract functions (according to consensus rules), update the distributed ledger with new transactions (according to the consensus rules), and manage copies of a distributed ledger 1002A-D. The distributed ledger 1002A-D contains transactions, uploaded data, and smart contracts grouped in sequences of blocks. Placement of smart contracts on the consensus nodes 1000A-D in the distributed ledger 1002A-D enables the smart contracts and their data to be maintained in a persistent state based on the state of the distributed ledger 1002A-D, and for transactions initiated by the smart contracts to be authorized based on consensus rules followed by the consensus nodes 1000A-D. In an exemplary configuration, the distributed ledger contains copies 1004A-D of a first smart contract for distributing trust assets (the trust assets and their ownership recorded in the distributed ledger 1002A-D) to beneficiaries and copies 1006A-D of a second smart contract for updating a car registry recorded in the distributed ledger 1002A-D.

In operation, a user node 1008 (for example a node at a law firm) deploys a copy of the first smart contract to a consensus node 1000A, which then shares the first smart contract with the other nodes. Once the first smart contract is added to the distributed ledger 1002A-D, a node at a bank 1010 may send a message to one of the consensus nodes 1000B to execute a function of the first smart contract to distribute a quantity of cryptocurrency (or other assets, as defined by the smart contract) to user accounts named in the first smart contract. To trigger the distribution, the consensus nodes 1000A-D will independently execute said function and communicate with one another to reach consensus about the correct result.

In another example of the ecosystem in operation, a node 1012 at a department of motor vehicles may transmit a message to a consensus node 1000A to execute a function of the second smart contract to upload updated car registry information to the distributed ledger 1002A-D. The distributed 1002A-D ledger is updated once the consensus nodes 1000A-D verify the message and reach consensus on the result of said function. A separate node 1014 (for example a node at a car dealership or a police department) may send a message with proper authenticating credentials to a consensus node 1000D to query and read a portion of the uploaded vehicle registry information from a copy 1002D of the distributed ledger.

In addition to the foregoing transactions related to smart contracts, a client wallet 1016 (for example a wallet present on a smart phone) may submit a transaction to a consensus node 1000C to transfer a quantity of cryptocurrency from one address to another address. The cryptocurrency transaction may be embedded into a smart contract function call whereby specified transactions may only occur if valid amounts of cryptocurrency are included in said transactions. Similarly to the previous transactions, prior to executing the transaction and updating the distributed ledger 1002A-D, the consensus nodes 1000A-D will receive and verify the transaction, followed by reaching consensus on the correct state of the distributed ledger 1002A-D.

All publications and patent applications mentioned in this specification are herein incorporated by reference to the same extent as if each individual publication or patent application was specifically and individually indicated to be incorporated by reference.

While preferred embodiments of the present invention have been shown and described herein, it will be obvious to those skilled in the art that such embodiments are provided by way of example only. It is intended that the following claims define the scope of the invention and that methods and structures within the scope of these claims and their equivalents be covered thereby.

1-27. (canceled)

28. A system for remote management of patient compliance, comprising:
a mobile medical device, comprising:
i) a processor;
ii) a non-transitory computer readable memory in communication with the processor;
iii) a data source in communication with the processor;
iv) a reference to predetermined network coordinates stored in the memory;
v) use instructions for treating a medical condition stored in the memory, and
vi) program code stored in the memory, the program code executable by the processor to perform data communication operations, the data communication operations comprising:
a) processing signals from the data source to obtain device data;
b) inserting dual payloads and device signatures applied to the dual payloads into network packets, first payloads of the dual payloads comprising at least portions of the device data, second payloads of the dual payloads comprising hashes of the at least portions of the device data and device signatures applied to the hashes; and
c) pushing the network packets to the predetermined network coordinates in response to internally-generated prompts.

29. The system of claim 28, wherein the system further comprises: a computing infrastructure comprising:
i) the predetermined network coordinates;
ii) a privacy-compliant database configured to receive the device data; and
iii) a list of public keys containing a public key for the mobile medical device, the public key for the mobile medical device effective to recover the hashes from the device signatures applied to the hashes; and
iv) at least one access point for communication with a distributed ledger network, the at least access point configured to push instructions to a distributed ledger to add the hashes to the distributed ledger without any patient identification information.

30. The system of claim 29, the computing infrastructure further comprising program code stored in a non-transitory computer readable memory, the program code executable by a processor to perform remote monitoring of the device data received by the privacy-compliant database.

31. The system of claim 30, wherein the remote monitoring comprises: adjusting a schedule for one or more of the pulling, downloading, and processing if the determined risk of readmission exceeds a first threshold; and optionally triggering an intervention protocol if the determined risk of readmission exceeds a second threshold.

32. The system of claim 30, wherein the remotely monitoring further comprises:
a) pulling the hash and the device signature applied to the hash from the distributed ledger;
b) authenticating the hash by recovering the hash from at least the device signature applied to the hash;
c) verifying the accuracy of the downloaded at least a portion of the device data against the pulled hash; and
d) processing the at least a portion of the device data in combination with the use instructions to obtain the at least one patient compliance parameter.

33. The system of claim 32, wherein the at least one patient compliance parameter comprises a measure of patient compliance with the use instructions.

34. The system of claim 33, wherein the at least one patient compliance parameter comprises:
a measure of patient non-compliance with the use instructions,
a cumulative number of missed treatments by the mobile medical device, a cumulative quantity of medication dispensed by the mobile medical device, a probability of readmission of the patient to a healthcare facility to treat the medical condition,
an estimate of an excess readmission rate, or any combination thereof.

35. The system of claim 28, wherein the dual payloads are recovered from the device signatures applied to the dual payloads using a public key.

36. The system of claim 35, wherein the public key is created by a manufacturer of the mobile medical device.

37. The system of claim 28, wherein the mobile medical device further comprises a private key embedded in an onboard key signing chip.

38. The system of claim 37, wherein the communication operations further comprise using the private key to generate the device signatures applied to the hashes.

39. The system of claim 28, wherein the second payloads contains no patient identification information.

40. The system of claim 28, wherein the second payload comprises one or more instructions for inserting the hash into a distributed ledger.

41. The system of claim 40, wherein the one or more instructions are recovered from the device signature applied to the hash.

42. The system of claim 29, wherein the computing infrastructure further comprises:
i) a further processor;
ii) a private key; and
iii) instructions executable by the further processor to use the private key to generate digital signatures applied to the device signatures applied to the hashes.

43. The system of claim 42, wherein the mobile medical device comprises a key signing chip that generates the private key.

44. The system of claim 28, wherein the device data comprises a log of mobile medical device usage data including one or more of time-of-use, frequency of use, and use-duration data for the mobile medical device.

45. The system of claim 44, wherein the log of mobile medical device usage data comprises at least one of time, duration, and frequency of usage of the mobile medical device.

46. The system of claim 28, wherein a first threshold is triggered when the log of mobile medical device usage indicates non-compliant usage, lack of usage, and/or overusage of the mobile medical device by the intended user.

47. The system of claim 28, comprising further mobile medical devices that are drug delivery devices, wherein the mobile medical device gathers and transmit data from the further mobile medical devices.

48. The system of claim 28, wherein the mobile medical device comprises a sensor or a dispenser.

49. The system of claim 48, wherein the mobile medical device comprises a mechanical actuator.

50. The system of claim 49, wherein the mechanical actuator is configured for operation by the patient.

51. The system of claim 28, wherein the mobile medical device is a nebulizer.
